(19) United States
(12) Patent Application Publication
(10) Pub. No.: US 2013/0131008 A1
(43) Pub. Date: May 23, 2013

(54) LIPOPHILIC MONOPHOSPHORYLATED DERIVATIVES AND NANOPARTICLES

(71) Applicant: Board of Regents, The University of Texas System, Austin, TX (US)

(72) Inventors: Zhengrong Cui, Austin, TX (US); Dharmika S. P. Lansakara-P., Austin, TX (US); Michael A. Sandoval, Austin, TX (US)

(73) Assignee: BOARD OF REGENTS, THE UNIVERSITY OF TEXAS SYSTEM, Austin, TX (US)

(21) Appl. No.: 13/660,217

(22) Filed: Oct. 25, 2012

Related U.S. Application Data
(60) Provisional application No. 61/551,262, filed on Oct. 25, 2011.

(51) Int. Cl. C07H 19/10

(52) U.S. Cl. CPC C07H 19/10
USPC 514/49; 536/26.8

(57) ABSTRACT

There are provided, inter alia, lipophilic monophosphorylated derivatives of gemcitabine. There are further provided nanoparticles compositions incorporating lipophilic monophosphorylated derivatives of gemcitabine, pharmaceutical compositions thereof, and a method of treating cancer or a viral infection in a subject in need thereof, which method includes administration of a pharmaceutical composition disclosed herein.

### CROSS-REFERENCES TO RELATED APPLICATIONS

This application claims the benefit of U.S. Provisional Application No. 61/551,262, filed Oct. 25, 2011, the entire contents of which is incorporated by reference herein and for all purposes.

### STATEMENT AS TO RIGHTS TO INVENTIONS MADE UNDER FEDERALLY SPONSORED RESEARCH AND DEVELOPMENT

This invention was made with government support under grant number R01 CA135274 awarded by the National Institutes of Health. The government has certain rights in the invention.

### BACKGROUND OF THE INVENTION

Gemcitabine HCl (2′,2′-difluorodeoxycytidine HCl, dFdC) is a clinically approved anticancer drug for the treatment of a wide spectrum of cancers including pancreatic, non-small-cell lung cancer, breast, bladder, head and neck, mesothelioma, cervical, and ovarian cancers. See e.g., Candelaria, M., et al., 2010, Med. Oncol. 27(4):1133-1143; Barton-Burke, M., 1999, Cancer Nurs. 22(2):176-183; Kalykaki, A., et al., 2008, Anticancer Res. 28(1B):495-500; Sandler, A. B., et al., 2000, J. Clin. Oncol. 18(1):122; Zucali, P. A., et al., 2008, Cancer 112(7):1555-1561; Cetina, L., et al., 2004, Anticancer Drugs 15(8):761-766; Candelaria, M., et al., 2007, Eur. J. Cancer Suppl. 5(1):37-43. However, tumor cells often acquire resistance during or after gemcitabine treatment. See e.g., Bergman, A. M., et al., 2002, Drug Resist. Updat. 5(1):19-33; Andersson, R., et al., 2009, Scand. J. Gastroenterol. 44(7):782-786; Sezgin, C., et al., 2005, Scand. J. Gastroenterol. 40(12):1486-1492. Gemcitabine is a polar deoxycytidine analogue. It requires nucleoside transporters to translocate across the cellular membrane. See e.g., Heinemann V., et al., 1995, Semin Oncol. 22:11-18. Clinical data showed that patients with tumors with a decreased expression of hENT1, a nucleoside transporter, have a significantly lower survival rate after gemcitabine treatment than those with tumors that express a higher level of hENT1. See e.g., Giovannetti, E., et al., 2006, Cancer Res. 66(7):3928-3935; Mey, V., et al., 2006, Br. J. Cancer 95(3):289-297; Spratlin, J., et al., 2004, Clin. Cancer Res. 10(20):6956-6961. More than 90% gemcitabine that are internalized into cells are deaminated by deoxycytidine deaminase (dCDA) to form inactive 2′-deoxy-2′,2′-difluorouridine (dFdU). Therefore, deamination affects the efficacy of gemcitabine adversely. See e.g., Immordino, M. L., et al., 2004, J. Control. Release 100(3):331-346. Gemcitabine is a prodrug. The term “prodrug” refers in the customary sense to a compound that readily undergo chemical changes under physiological conditions to provide a benefit. Specifically gemcitabine needs to be phosphorylated to gemcitabine monophosphate (dFdCMP) by dCK. See e.g., Kroep, J. R., et al., 2002, Mol. Cancer. Ther. 1(6):371-376; Bouffard, D. Y., et al., 1993, Biochem. Pharmacol. 45(9):1857-1861. Subsequently, dFdCMP is phosphorylated by nucleotide kinases to di- and tri-phosphorylated gemcitabine (dFdCDP and dFdCTP, respectively) that are active metabolites of gemcitabine. See e.g., Bergman, A. M., et al., 2002, Drug Resist. Updat. 5(1):19-33; Mini, E., et al., 2006, Ann. Oncol. 17:v7-v12; Ueno, H., et al., 2007, Br. J. Cancer 97(2):145-151. Therefore, tumor cells that are deficient in dCK are resistant to gemcitabine. Clinical studies in patients with resected pancreatic adenocarcinoma showed a strong correlation between low level of dCK expression and poor clinical outcomes after gemcitabine-based adjuvant therapy. See e.g., Maréchal, R., et al., 2010, Cancer 116(22):5200-5206. Disease-free survival was significantly longer in patients having high levels of dCK expression (38.6-77.5 months) than in patients having low levels of dCK expression (2.9-9.6 months). See Maréchal, R., et al., 2010, Id. The antipoliferative activity of gemcitabine is known to be exerted mainly through the inhibition of DNA synthesis by masked chain termination and inhibition of DNA polymerase by dFdCTP. See e.g., Huang, P. & Plunkett, W., 1995, Semin Oncol. 4(11):19-25. Ribonucleotide reductase (RR) is required to convert ribonucleotides to deoxyribonucleotides. dFdCDP inhibits RR, leading to the depletion of deoxynucleotide triphosphate (dNTP) pool and the enhancement of the activity of gemcitabine. See e.g., Heinemann, V., et al., 1992, Cancer Res. 52(3):533-539. Active RR composes of two homodimers of non-identical subunits, the large RRM1 subunit and the small RRM2 subunit. See e.g., Candelaria, M., et al., 2010, Id. Both pre-clinical and clinical data have shown that tumor cells that over-express of RRM1 or RRM2 are resistant to gemcitabine. See e.g., Jordheim, L. P., et al., 2005, Mol. Cancer. Ther. 4(8):1268-1276; Boukovinas, I., et al., 2008, PLoS. ONE 3(11):e3695.

The most commonly reported acquired resistance to gemcitabine is dCK deficiency. See e.g., Bergman, A. M., et al., 2002, Id.; Gregoire, V., et al., 2002, Radiother. Oncol. 63:329-338. Inefficient intracellular monophosphorylation of gemcitabine may reduce the efficacy of gemcitabine drastically.

Thus, there is a need in the art for new gemcitabine analogs that are effective, for example, in gemcitabine resistant subjects for cancer treatment and treatment of viral infections. The present invention addresses these and other needs in the art.

### BRIEF SUMMARY OF THE INVENTION

In a first aspect, there is provided a compound with structure of Formula (I)

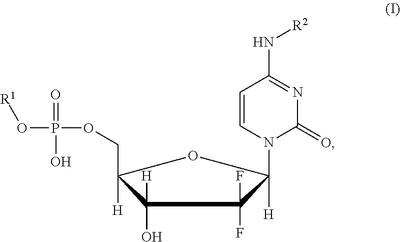

or pharmaceutically acceptable salt thereof. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In another aspect, there is provided a nanoparticle composition which includes a compound having the structure of Formula (I) or pharmaceutically acceptable salt thereof. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In another aspect, there is provided a pharmaceutical composition including a compound with the structure of Formula (I) or pharmaceutically acceptable salt thereof, and a pharmaceutically acceptable excipient. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In another aspect, there is provided a method of treating cancer or a viral infection in a subject in need thereof. The method includes administering to the subject a therapeutically effective amount of a compound as disclosed herein, a nanoparticle composition as disclosed herein, or a pharmaceutical composition as disclosed herein.

### BRIEF DESCRIPTION OF THE DRAWINGS

FIG. 1 depicts % PANC-1 cells alive after incubation with gemcitabine HCl (GEM) at 400 μM for up to 116 h. Cell viability was determined using a trypan blue exclusion assay. Data shown are mean±S.D. (S.D.: standard deviation) (n=3). Experiment was repeated twice.

FIG. 2 depicts dCDA assay. (FIG. 2A) Effect of gemcitabine concentration on the conversion of dCyd to dUrd (ratio, w/w) by dCDA. (FIG. 2B) Effect of GemC18-NPs and 8-NPs on the conversion of dCyd to dUrd by dCD (No, no inhibitor; GEM, gemcitabine HCl, 1.7 mM). The molar concentration of GemC18 and 8 in the nanoparticles was 1.7 mM. Blank nanoparticles did not inhibit the conversion. Data shown are mean±S.D. The experiment was repeated at least twice with similar results.

FIG. 3 depicts inhibition of TC-1 tumor growth upon oral administration of Cmpd 8. Legend: Mannitol control (open box); Cmpd 8 in oil (closed triangle); Cmpd 8 nanoparticle (open triangle); Cmpd 8 nanoparticle (I.V.) (closed box).

FIGS. 4A-4B depict plasma gemcitabine concentration (μg/mL) at time (h) after GemC18-NPs were intravenously injected (FIG. 4A) or orally gavaged (FIG. 4B) into mice. See Example 18.

FIG. 5 depicts tumor diameter over time (days, d) in TC-1 tumor-bearing C57BL/6 mice after s.c. injection twice per week of control or test compound. Legend: untreated (triangle); PLGA-NP (gray circle); PEG-PLGA-GemC18-NP (black circle); gemcitabine (open box). See Example 19.

FIG. 6 depicts the time course (days, d) of the effect on tumor volume (cm³) of normal saline, gemcitabine HCl (GemHCl) solution, GemC18 solution, blank PEG-C18 micelles, and GemC18/PEG-C18 micelles on the growth of B16-F10 melanoma tumor in mice (n=5-8). Arrow indicated days of injection. a, p<0.05, GemC18/PEG-C18 micelles vs. saline starting on day 2; b, p<0.05, GemC18/PEG-C18 micelles vs. GemHCl solution starting on day 3; c, p<0.05, GemC18/PEG-C18 micelles vs. GemC18 solution starting on day 5. Legend: Saline (closed triangle); GemHCl solution (open triangle); GemC18 solution (diamond); Blank PEG-C18 micelles (closed box); GemC18/PEG-C18 micelles (open box).

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butyryl, and the higher homologs and isomers. An alkoxy is an alkyl attached to the remainder of the molecule via an oxygen linker (—O—). An alkylthiol is an alkyl attached to the remainder of the molecule via a sulfur linker (—S—).

The term “alkylene,” by itself or as part of another substituent, means, unless otherwise stated, a divalent radical derived from an alkyl, as exemplified but not limited by, —CH₂—, —CH₂CH₂—, —CH₂CH₂CH₂—, —CH₂CH₂CH₂CH₂—, and the like. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided herein.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are as disclosed herein or can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2 m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are described herein or are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

Unless otherwise stated, a “substituent group” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₅-C₁₀ aryl, and each substituted or unsubstituted heteroaryl is a substituted or unsubstituted C₅-C₁₀ heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₁₀ alkyl, each substituted or unsubstituted heteroalkyl is a substituted or unsubstituted 2 to 10 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₅-C₁₀ (e.g., C₅-C₆) aryl, and each substituted or unsubstituted heteroaryl is a substituted or unsubstituted C₅-C₁₀ (e.g., C₅-C₆) heteroaryl.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope contemplated herein.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon are within the scope contemplated herein.

The compounds disclosed herein may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope contemplated herein.

The term “nanoparticle” as used herein refers to an assemblage as known in the art generally having a longest dimension of about 1000 nm or less. In some embodiments, the longest dimension is about 100 nm or less, or about 10 nm or less. In some embodiments, the nanoparticle has a longest dimension of about 10-1000 nm, 10-100 nm, 50-1000 nm, 50-900 nm, 50-800 nm, 50-700 nm, 50-600 nm, 50-500 nm, 50-400 nm, 50-300 nm, 50-200 nm, 50-100 nm, 100-1000 nm, 100-900 nm, 100-800 nm, 100-700 nm, 100-600 nm, 100-500 nm, 100-400 nm, 100-300 nm, 100-290 nm, 100-280 nm, 100-270 nm, 100-260 nm, 100-250 nm, 100-240 nm, 100-230 nm, 100-220 nm, 100-210 nm, 100-200 nm, 125-500 nm, 125-400 nm, 125-300 nm, 125-290 nm, 125-280 nm, 125-270 nm, 125-260 nm, 125-250 nm, 125-240 nm, 125-230 nm, 125-220 nm, 125-210 nm, 125-200 nm, 125-190 nm, 125-180 nm, 125-175 nm, 125-120 nm, 125-165 nm, 125-160 nm, 125-155 nm, or 125-150 nm. In some embodiments, the longest dimension is about 5, 10, 20, 30, 40, 50, 60, 70, 80, 90, 100, 110, 120, 130, 140, 142, 144, 146, 148, 150, 152, 154, 156, 158, 160, 162, 164, 166, 168, 170, 180, 190, or 200 nm. Absent express indication otherwise, the term “about” in the context of a numeric value refers to the nominal numeric value +/−10% thereof. Nanoparticles can be held together by covalent or non-covalent forces, as understood in the art.

Exemplary nanoparticles include a variety of types known in the art, e.g., solid, semi-solid, and soft nanoparticles. Typical solid nanoparticles include solid lipid nanoparticles as disclosed herein. Typical semi-solid nanoparticle include liposomes, as known in the art. Typical soft nanoparticles include nanogel particles as known in the art, which are formed by polymeric chains loosely cross-linked to form a three-dimensional network, as known in the art. Poly(lactic acid) (PLA), Poly(glycolic acid) (PGA), and their copolymers (PLGA) have been extensively investigated in the preparation of microparticles and nanoparticles. See e.g., U.S. Pat. No. 7,563,871, incorporated herein by reference in its entirety and for all purposes. In some embodiments, the nanoparticle is a micelle, as known in the art.

Methods of forming nanoparticles of the compounds disclosed herein can include formation of a homogenous slurry in the presence of a lipid component, e.g., soy lecithin. A detergent, e.g., Tween™ 20, can be added in a stepwise manner. The resultant emulsion can then be cooled, e.g., to room temperature, while stirring to form nanoparticles. Nanoparticle physical properties, e.g., particle size (e.g., longest dimension), polydispersity, and zeta potential, can be measured by methods known in the art. Other methods of forming micelles and nanoparticles are described herein or are known in the art.

The term “microparticle” as used herein refers to an assemblage as known in the art generally having a longest dimension of about 1 μm to about 100 μm. In some embodiments, the microparticle has a longest dimension of about 1-100 μm, 2-100 μm, 5-100 μm, 10-100 μm, 20-100 μm, 30-100 μm, 40-100 μm, 50-100 μm, 60-100 μm, 70-100 μm, 80-100 μm, 90-100 μm, 1-10 μm, 2-10 μm, 3-10 μm, 4-10 μm, 5-10 μm, 10-20 μm, 10-30 μm, 10-40 μm, 10-50 μm, 10-60 μm, 10-70 μm, 10-80 μm, or 10-90 μm. Microparticle can be held together by covalent or non-covalent forces, as understood in the art. Methods for form microparticles includes methods known in the art.

Nanoparticles and microparticles can be characterized by a variety of physical parameters. For example, “polydispersity index” and “PI” refer to a parameter defining a nanoparticle size distribution obtained, e.g., from photon correlation spectroscopic studies. It is a dimensionless number extrapolated from the autocorrelation function and ranges from a value of about 0.01 for monodisperse nanoparticle samples up to values of about 0.50 to 0.70 for more polydisperse samples. Samples with very broad size distribution have PI values greater than about 0.70. For microparticles, methods for determination of particle size include optical and electron microscopy (e.g., TEM, SEM), as known in the art.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating,” “treatment” and the like refer to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods presented herein successfully treat cancer by decreasing the incidence of cancer and/or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof include prevention of an injury, pathology, condition, or disease.

The term “patient,” “subject,” “subject in need thereof” and the like refer to a living organism suffering from or prone to a disease or condition that can be treated by administration of a compound or pharmaceutical composition as provided herein. Non-limiting examples include humans, other mammals, bovines, rats, mice, dogs, monkeys, goat, sheep, cows, deer, and other non-mammalian animals. In one embodiment, a patient is human.

The term “cancer” refers to all types of cancer, neoplasm or malignant tumors found in mammals (e.g., humans), including leukemia, carcinomas and sarcomas. Exemplary cancers that may be treated with a compound or method provided herein include cancer of the thyroid, endocrine system, brain, breast, bladder, cervix, colon, head and neck, liver, kidney, lung, non-small cell lung, melanoma, mesothelioma, ovary, sarcoma, stomach, uterus, Medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In one embodiment, compounds and compositions disclosed herein are useful in the treatment of pancreatic cancer, non-small-cell lung cancer, breast cancer, bladder cancer, cancers of the head and neck, mesothelioma, cervical cancer, or ovarian cancer.

### II. Compounds

In a first aspect, there is provided a compound with structure of Formula (I)

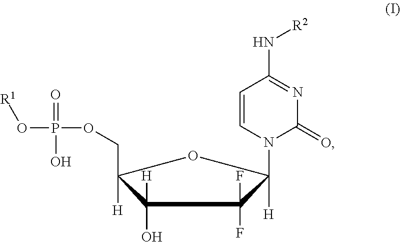

or pharmaceutically acceptable salt thereof. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In some embodiments, R¹ is hydrogen. In some embodiments, R² is hydrogen.

In some embodiments, R¹ is unsubstituted C₁₂-C₂₄ alkyl, e.g., C₁₂, C₁₃, C₁₄, C₁₅, C₁₆, C₁₇, C₁₈, C₁₉, C₂₀, C₂₁, C₂₂, C₂₃, or C₂₋₄ alkyl, preferably C₁₋₈ alkyl. In some embodiments R¹ is unsubstituted C₁₂-C₂₀ alkyl. In some embodiments R¹ is unsubstituted C₁₆-C₂₀ alkyl. In some embodiments R¹ is unsubstituted C₁₆-C₁₈ alkyl. In some embodiments R¹ is unsubstituted C₁₈ alkyl. In some embodiments, R¹ is an unbranched C₁₂-C₂₄ alkyl, unbranched C₁₂-C₂₀ alkyl, unbranched C₁₆-C₂₀ alkyl or unbranched C₁₆-C₁₈ alkyl, preferably unbranched C₁₋₈ alkyl. In some embodiments, R¹ is an branched C₁₂-C₂₄ alkyl, branched C₁₂-C₂₀ alkyl, branched C₁₆-C₂₀ alkyl or branched C₁₆-C₁₈ alkyl.

In some embodiments, the compound has the structure of Formula (Ia):

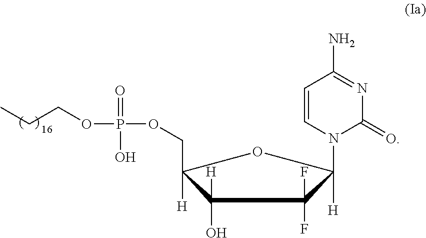

In some embodiments, the compound has the structure of Formula (I) wherein R¹ is substituted or unsubstituted C₁₂-C₂₇ heteroalkyl. In some embodiments, R¹ is R⁴—CO—O-L¹-, and the compound has the structure of Formula (I-1):

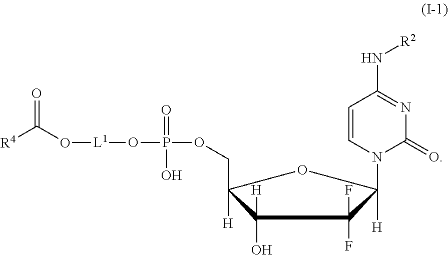

Further to compounds with the structure of Formula (I-1), in some embodiments R⁴ is substituted or unsubstituted C₁₀-C₂₀ alkyl. In some embodiments, R⁴ is unsubstituted C₁₀, C₁₁, C₁₂, C₁₃, C₁₄, C₁₅, C₁₆, C₁₇, C₁₈, C₁₉ or C₂₀ alkyl. In some embodiments, R⁴ is C₁₀, C₁₁, C₁₂, C₁₃, C₁₄, C₁₅, C₁₆, C₁₇, C₁₈, C₁₉ or C₂₀ alkyl substituted with one or more of —OH, —NH₂, —SH, —CN, —CF₃, —NO₂, oxo, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹ is substituted or unsubstituted C₁-C₆ alkylene. In some embodiments, L¹ is unsubstituted C₁-C₆ alkylene. In some embodiments, L¹ is unsubstituted C₁, C₂, C₃, C₄, C₅ or C₆ alkylene. In some embodiments, L¹ is R⁵-substituted C₁-C₆ alkylene. R⁵ is —OH, —NH₂, —SH, —CN, —CF₃, —NO₂, oxo, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁵ is —OH, —NH₂, —SH, —CN, —CF₃, —NO₂, oxo, or halogen. In some embodiments, R⁵ is halogen. In some embodiments, R⁵ is chloro. In some embodiments, L¹ is R⁵— substituted C₃ alkylene, and R⁵ is halogen.

In one embodiment, the compound has the structure of Formula (Ib):

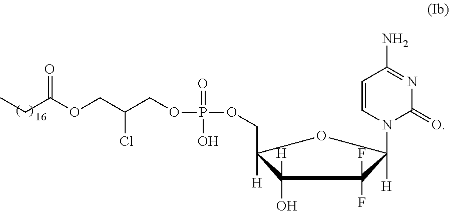

In some embodiments of the compound with structure of Formula (I), R² is —CO—R³. In some embodiments, R¹ is unsubstituted C₁₂-C₂₄ alkyl, e.g., C₁₂, C₁₃, C₁₄, C₁₅, C₁₆, C₁₇, C₁₈, C₁₉, C₂₀, C₂₁, C₂₂, C₂₃, or C₂₄ alkyl, preferably C₁₈ alkyl. In some embodiments, R³ is unsubstituted C₁-C₂₄ alkyl, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl. In some embodiments, R³ is substituted C₁-C₂₄ alkyl, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄ alkyl substituted with one or more of —OH, —NH₂, —SH, —CN, —CF₃, —NO₂, oxo, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, the compound has the structure of Formula (Ic):

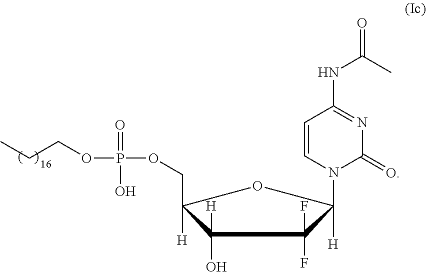

In some embodiments of the compound with structure of Formula (I), R¹ is hydrogen. In some embodiments, the compound has the structure of Formula (Id):

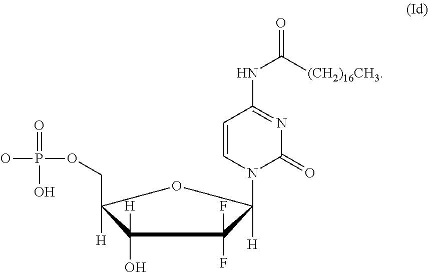

In one embodiment, R¹ is R⁶-substituted C₁₂-C₂₇ heteroalkyl. R⁶ is independently halogen, —CN, —CF₃, —OH, —NH₂, —SO₂, —COOH, R⁷-substituted or unsubstituted alkyl, R⁷-substituted or unsubstituted heteroalkyl, R⁷-substituted or unsubstituted cycloalkyl, R⁷-substituted or unsubstituted heterocycloalkyl, R⁷-substituted or unsubstituted aryl, or R⁷-substituted or unsubstituted heteroaryl. R⁷ is independently halogen, —CN, —CF₃, —OH, —NH₂, —SO₂, —COOH, R⁸-substituted or unsubstituted alkyl, R⁸-substituted or unsubstituted heteroalkyl, R⁸-substituted or unsubstituted cycloalkyl, R⁸-substituted or unsubstituted heterocycloalkyl, R⁸-substituted or unsubstituted aryl, or R⁸-substituted or unsubstituted heteroaryl. R⁸ is independently halogen, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In one embodiment, R⁶, R⁷ and/or R⁸ is a size-limited substituent group or a lower substituent group. In one embodiment, R⁶ and R⁷ are independently halogen, —CN, —CF₃, —OH, —NH₂, —COOH, substituted or unsubstituted C₁-C₁₀ (e.g., C₁-C₆) alkyl, substituted or unsubstituted 2 to 10 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In some embodiments, R⁸ is halogen, —CN, —CF₃, —OH, —NH₂, —COOH, unsubstituted C₁-C₁₀ (e.g., C₁-C₆) alkyl, unsubstituted 2 to 10 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In one embodiment, R³ is R⁹-substituted C₁-C₂₄ alkyl. R⁹ is independently halogen, —CN, —CF₃, —OH, —NH₂, —SO₂, —COOH, R¹⁰-substituted or unsubstituted alkyl, R¹⁰-substituted or unsubstituted heteroalkyl, R¹⁰-substituted or unsubstituted cycloalkyl, R¹⁰-substituted or unsubstituted heterocycloalkyl, R¹⁰-substituted or unsubstituted aryl, or R¹⁰-substituted or unsubstituted heteroaryl. R¹⁰ is independently halogen, —CN, —CF₃, —OH, —NH₂, —SO₂, —COOH, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. R¹¹ is independently halogen, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In one embodiment, R⁹, R¹⁰ and/or R¹¹ is a size-limited substituent group or a lower substituent group. In one embodiment, R⁹ and R¹⁰ are independently halogen, —CN, —CF₃, —OH, —NH₂, —COOH, substituted or unsubstituted C₁-C₁₀ (e.g., C₁-C₆) alkyl, substituted or unsubstituted 2 to 10 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In some embodiments, R¹¹ is halogen, —CN, —CF₃, —OH, —NH₂, —COOH, unsubstituted C₁-C₁₀ (e.g., C₁-C₆) alkyl, unsubstituted 2 to 10 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In one embodiment, L¹ is R¹²-substituted C₁-C₆ alkylene. R¹² is independently halogen, —CN, —CF₃, —OH, —NH₂, —SO₂, —COOH, R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or R¹³-substituted or unsubstituted heteroaryl. R¹³ is independently halogen, —CN, —CF₃, —OH, —NH₂, —SO₂, —COOH, R¹⁴-substituted or unsubstituted alkyl, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or R¹⁴-substituted or unsubstituted heteroaryl. R¹⁴ is independently halogen, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In one embodiment, R¹², R¹³ and/or R¹⁴ is a size-limited substituent group or a lower substituent group. In one embodiment, R¹² and R¹³ are independently halogen, —CN, —CF₃, —OH, —NH₂, —COOH, substituted or unsubstituted C₁-C₁₀ (e.g., C₁-C₆) alkyl, substituted or unsubstituted 2 to 10 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In some embodiments, R¹⁴ is halogen, —CN, —CF₃, —OH, —NH₂, —COOH, unsubstituted C₁-C₁₀ (e.g., C₁-C₆) alkyl, unsubstituted 2 to 10 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In one embodiment, R⁴ is R¹⁵-substituted C₁₀-C₂₀ alkyl. R¹⁵ is independently halogen, —CN, —CF₃, —OH, —NH₂, —SO₂, —COOH, R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl. R¹⁶ is independently halogen, —CN, —CF₃, —OH, —NH₂, —SO₂, —COOH, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. R¹⁷ is independently halogen, —CN, —CF₃, —OH, —NH₂, —SO₂, —COOH, unsubstituted alkyl, unsubstituted heteroalkyl, unsubstituted cycloalkyl, unsubstituted heterocycloalkyl, unsubstituted aryl, or unsubstituted heteroaryl. In one embodiment, R¹⁵, R¹⁶ and/or R¹⁷ is a size-limited substituent group or a lower substituent group. In one embodiment, R¹⁵ and R¹⁶ are independently halogen, —CN, —CF₃, —OH, —NH₂, —COOH, substituted or unsubstituted C₁-C₁₀ (e.g., C₁-C₆) alkyl, substituted or unsubstituted 2 to 10 membered (e.g., 2 to 6 membered) heteroalkyl, substituted or unsubstituted C₃-C₈ (e.g., C₅-C₇) cycloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl. In one embodiment, R¹⁷ is halogen, —CN, —CF₃, —OH, —NH₂, —COOH, unsubstituted C₁-C₁₀ (e.g., C₁-C₆) alkyl, unsubstituted 2 to 10 membered (e.g., 2 to 6 membered) heteroalkyl, unsubstituted C₃-C₈ (e.g., C₅-C₇) cycloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In another aspect, there is provided a microparticle composition which includes a compound having the structure of Formula (I) or pharmaceutically acceptable salt thereof. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In some embodiments, the microparticles and nanoparticles disclosed herein have an interior region composed essentially of lipid, which interior region is enclosed by a hydrophilic outer layer. Certain components of the microparticles and nanoparticles may be amphiphilic and positioned to provide the interior region composed essentially of lipid and the hydrophilic outer layer. Microparticles and nanoparticles which incorporate the compounds disclosed herein can be prepared by, e.g., mixing soy lecithin and optionally glycerol monostearate with the compound in water at moderate temperature (e.g., 90-105° C.) to form a homogeneous slurry. To this slurry can be added an amphiphilic emulsifier (e.g., Tween™ 20) with stirring. The resulting emulsion can be allowed to cool, resulting in the formation of microparticles or nanoparticles. Accordingly, the lipids contained within the interior region of the microparticle or nanoparticle may derive from the components used in formation of the microparticle or nanoparticle. In some embodiments, the compounds disclosed herein are associated with the resulting microparticles or nanoparticles by virtue of a lipophilic functionality of the compound (e.g., R¹ and/or R² of compounds of Formula I) which can embed in the lipid interior region of the microparticle or nanoparticle. Accordingly, the terms “include,” “includes a compound” and the like in the context of the association between the compounds disclosed herein and microparticles or nanoparticles may, in some embodiments, refer to an anchoring of the compound in the microparticle or nanoparticle such that a lipophilic functionality resides in the interior region composed essentially of lipid, and the non-lipophilic functionalities (e.g., nucleobase, substituted sugar, phosphate moiety, and the like) reside outside of the interior region.

In some embodiments, the microparticles have a longest dimension of about 1-100 μm, 2-100 μm, 5-100 μm, 10-100 μm, 20-100 μm, 30-100 μm, 40-100 μm, 50-100 μm, 60-100 μm, 70-100 μm, 80-100 μm, 90-100 μm, 1-10 μm, 2-10 μm, 3-10 μm, 4-10 μm, 5-10 μm, 10-20 μm, 10-30 μm, 10-40 μm, 10-50 μm, 10-60 μm, 10-70 μm, 10-80 μm, or 10-90 μm.

In some embodiments, the microparticle composition includes a compound having the structure of any one of Formulae (Ia), (Ib), (Ic) or (Id) disclosed herein.

In another aspect, there is provided a nanoparticle composition which includes a compound having the structure of Formula (I) or pharmaceutically acceptable salt thereof. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In some embodiments, the nanoparticles have a longest dimension of about 10-1000 nm, 10-100 nm, 50-1000 nm, 50-900 nm, 50-800 nm, 50-700 nm, 50-600 nm, 50-500 nm, 50-400 nm, 50-300 nm, 50-200 nm, 50-100 nm, 100-1000 nm, 100-900 nm, 100-800 nm, 100-700 nm, 100-600 nm, 100-500 nm, 100-400 nm, 100-300 nm, 100-290 nm, 100-280 nm, 100-270 nm, 100-260 nm, 100-250 nm, 100-240 nm, 100-230 nm, 100-220 nm, 100-210 nm, 100-200 nm, 125-500 nm, 125-400 nm, 125-300 nm, 125-290 nm, 125-280 nm, 125-270 nm, 125-260 nm, 125-250 nm, 125-240 nm, 125-230 nm, 125-220 nm, 125-210 nm, 125-200 nm, 125-190 nm, 125-180 nm, 125-175 nm, 125-120 nm, 125-165 nm, 125-160 nm, 125-155 nm, or 125-150 nm. In some embodiments, the longest dimension is less than about 1000 nm, 900 nm, 800 nm, 700 nm, 600 nm, 500 nm, 400 nm, 300 nm, 200 nm, or 100 nm. In some embodiments, the longest dimension is about 100-300 nm, 100-250 nm, 125-250 nm, 125-200 nm, 125-175 nm, or 150-200 nm. In some embodiments, the longest dimension is about 5, 10, 20, 30, 40, 50, 60, 70, 80, 90, 100, 110, 120, 130, 140, 142, 144, 146, 148, 150, 152, 154, 156, 158, 160, 162, 164, 166, 168, 170, 180, 190, or 200 nm.

In some embodiments, the polydispersity index (PI) of the nanoparticle composition is in the range of about 0.01-0.70, 0.02-0.70, 0.03-0.70, 0.04-0.70, 0.05-0.70, 0.06-0.70, 0.07-0.70, 0.08-0.70, 0.09-0.70, 0.10-0.70, 0.10-0.60, 0.10-0.50, 0.10-0.40, 0.10-0.30, 0.10-0.20, 0.20-0.70, 0.20-0.60, 0.20-0.50, 0.20-0.40, 0.20-0.30, 0.30-0.70, 0.30-0.60, 0.30-0.50, or 0.30-0.40. In some embodiments, the PI of the nanoparticle composition is in the range of about 0.20-0.40, 0.22-0.40, 0.24-0.40, 0.26-0.40, 0.28-0.40, 0.30-0.40, 0.32-0.40, 0.34-0.40, 0.36-0.40 or 0.38-0.40. In some embodiments, the polydispersity index is about 0.20, 0.22, 0.24, 0.26, 0.28, 0.30, 0.32, 0.34, 0.36, 0.38 or 0.40.

In some embodiments, the zeta potential, as known in the art, of the nanoparticle composition is in the range of about −100 to 100 mV, −90 to 90 mV, −80 to 80 mV, −70 to 70 mV, −60 to 60 mV, −50 to 50 mV, −40 to 40 mV, −30 to 30 mV, −20 to 20 mV, −10 to 10 mV, −100 to zero mV, −90 to zero mV, −80 to zero mV, −70 to zero mV, −60 to zero mV, −50 to zero mV, −40 to zero mV, −30 to zero mV, −20 to zero mV, −10 to zero mV. In some embodiments, the zeta potential is about −100 to −10 mV, −90 to −10 mV, −80 to −10 mV, −70 to −10 mV, −60 to −10 mV, −50 to −10 mV, −40 to −10 mV, −30 to −10 mV or −20 to −10 mV. In some embodiments, the zeta potential is about −50.0 to −20.0 mV, −45.0 to −20.0 mV, −40.0 to −20.0 mV, −35.0 to −20.0 mV, or −30.0 to −20.0 mV. In some embodiments, the zeta potential is about −50, −45, −44, −43, −42, −41, −40, −39, −38, −37, −36, −35, −34, −33, −32, −31, or −30 mV. In some embodiments, the size of the nanoparticles forming the nanoparticle composition is about 150-175 nm, with a polydispersity index of about 0.2-0.3, and a zeta potential of about −27 mV to −40 mV.

In some embodiments, the nanoparticle composition includes a compound having the structure of any one of Formulae (Ia), (Ib), (Ic) or (Id) following:

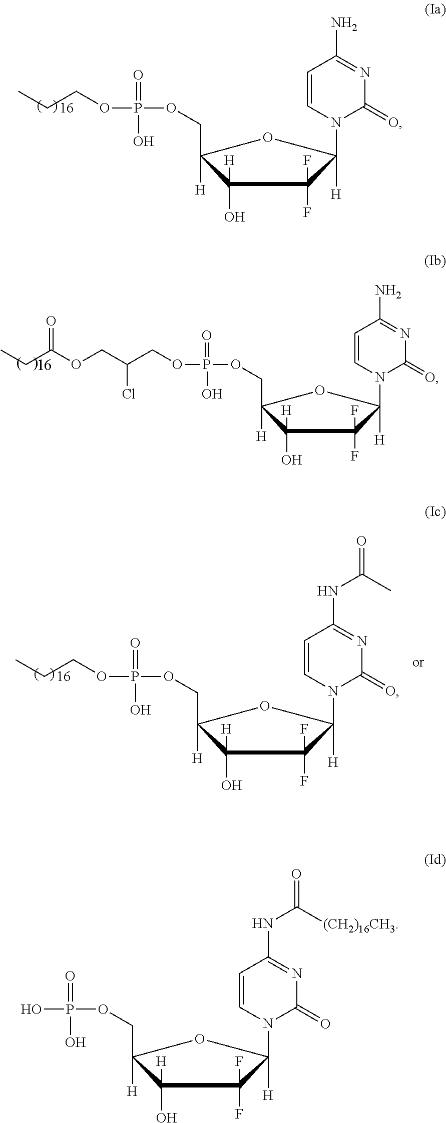

### A. Exemplary Synthesis

The compounds disclosed herein can be synthesized by an appropriate combination of generally well known synthetic methods. For example, GemC18 was synthesized according to literature procedures with slight modifications (Scheme 1). See e.g., Immordino, M. L., et al., 2004, Id.; Sloat, B. R., et al., 2011, Int. J. Pharm. 409(1-2):278-288; Guo, Z.-w. & Gallo, J. M., 1999, J. Org. Chem. 64(22):8319-8322. Briefly, the primary and secondary alcohols of deoxyribofuranose ring of gemcitabine (1) were Boc (tert-butoxycarbonyl) protected to prevent potential side reactions. The stearoyl group was conjugated to 4-amino group by reacting stearic acid, 1-hydroxy-7-azabenzotriazole (HOAt), and 1-ethyl-3-(3-dimethylaminopropyl) carbodiimide (EDCI) with 2 in anhydrous dichloromethane (DCM) for 30 h at ambient temperature. The Boc groups were removed using trifluoroacetic acid (TFA) in DCM to obtain 4 or GemC18 as a white crystalline powder (Steps a-c; Scheme 1). Reagents and conditions for Scheme 1: (a) Boc₂O, KOH, 1,4 dioxane, 22° C.; (b) CH₃(CH₂)₁₆COOH, EDCI, HOAt, DCM, rt; (c) TFA, DCM, rt; (d) i. POCl₃, triethylamine, DCM, reflux 2 h; ii. NaHCO₃, rt, 15 h; (e) Boc₂O, Na₂CO₃, dioxane, H₂O; (f) Boc₂O, dioxane, 37° C., 250 rpm, 72 h; (g) 6, TPS, pyridine, 38-40° C., 24 h; (h) TFA, DCM; (i) Acetic anhydride, CH₃OH, reflux; (j) 10, TPS, pyridine, 38-40° C., 24 h; (k) TFA, DCM.

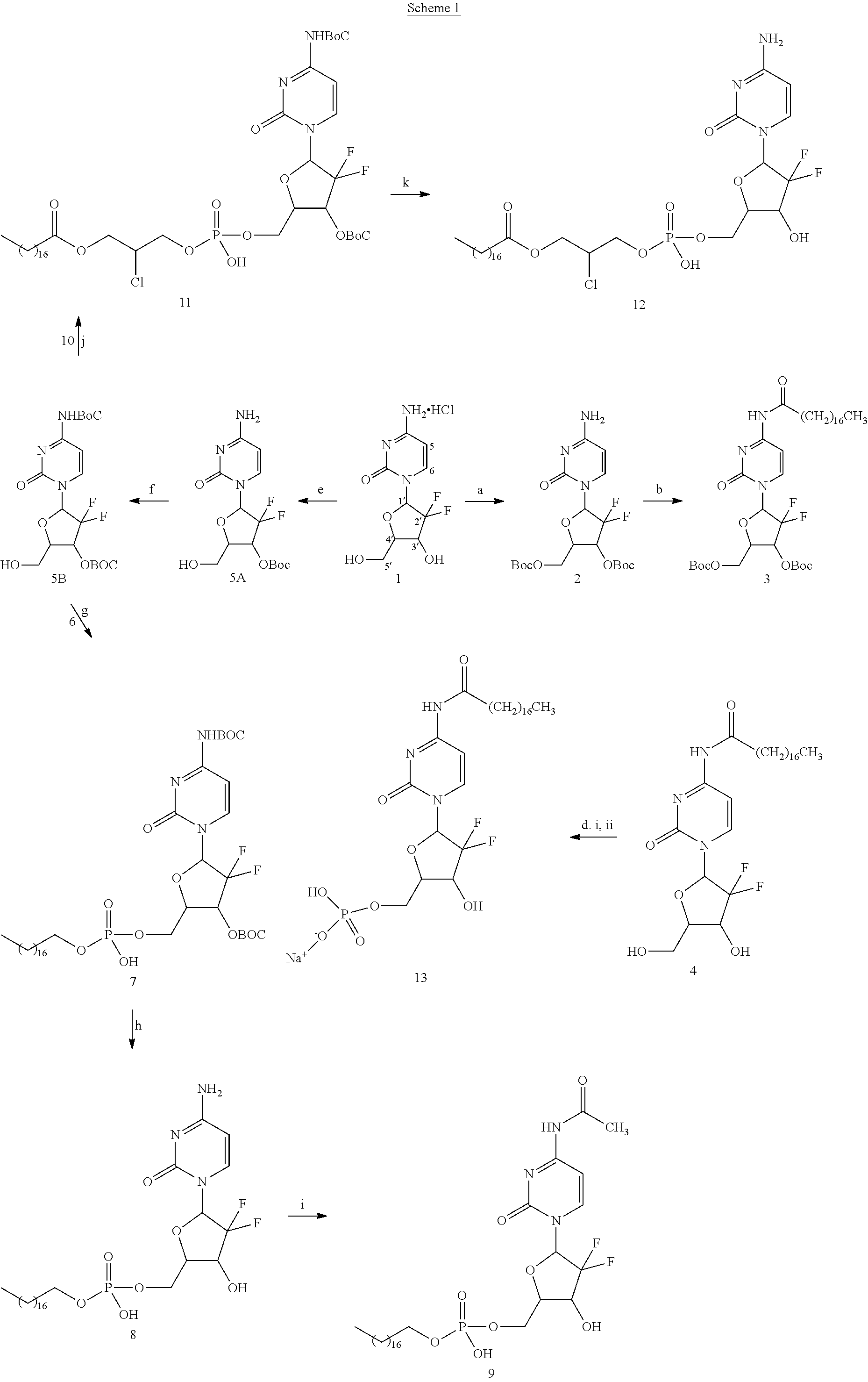

To facilitate direct conjugation to the 5′-OH, the primary alcohol and 4-amino groups of gemcitabine were Boc protected (5B). See e.g., Guo, Z.-w. & Gallo, J. M., 1999, Id. Octadecanol was phosphorylated by refluxing with phosphorus oxychloride (POCl₃) and triethylamine in DCM under argon, followed by the addition of sodium bicarbonate (NaHCO₃) and acidification to give the desired product of octadecylphosphate (6). See e.g., Bligh, E. G. & Dyer, W. J., 1959, Can. J. Biochem. Physiol. 37:911-917; Perie, J., et al., 1990, FR. Patent 2636331. The mixture of lyophilized powders of 5B and 6 were conjugated at the 5′-OH by the addition of 2,4,6-triisopropylbenzenesulfonyl chloride (TPS) in anhydrous pyridine under argon environment. The Boc groups were removed using TFA, and the crude sample was chromatographed on silica gel by eluting with 20%, 40%, and 50% methanol (CH₃OH) in chloroform (CHCl₃), sequentially, to obtain 2′-2′-difluoro-deoxycytidine-5′-octadecylphosphate (8). Acetylation of 8 on the 4-amino group was achieved by refluxing it with acetic anhydride in methanol (CH₃OH) to obtain compound 9 (Steps e-i; Scheme 1). See e.g., Watanabe, K. A. & Fo, J. J., 1966, J. Angew. Chem., Int. Ed. 5(6):579-580.

Glycerol monostearate was phosphorylated according to literature procedures to obtain 1-stearate-2-hydroxy-3-phosphatidic acid (10). See e.g., Bligh, E. G. & Dyer, W. J., 1959, Id.; Perie, J., et al., 1990, Id. The 5B and 10 were conjugated by the addition of TPS in anhydrous pyridine under argon environment. See e.g., Alexander, R. L., et al., 2003, J. Med. Chem. 46(19):4205-4208. Deprotection of Boc groups by the addition of TFA resulted in 2′-2′-difluoro-2′-deoxycytidine 5′(3-stearoyloxy-2-rac-chloropropyl)phosphate (12) (Steps j-k; Scheme 1).

GemC18 was phosphorylated by refluxing with POCl₃ and triethylamine in DCM. The reaction mixture was filtered to remove triethylamine hydrochloride, and the filtrate was added to NaHCO₃. The precipitate was washed with acetone, re-dissolved in water, and precipitated out again by the addition of acetone. 4-N-Octadecanoyl-2′,2′-difluoro-2′-deoxycytidine 5′-sodium phosphate salt (13) was isolated by filtration, washing several times with acetone, and drying under vacuum (Step d; Scheme 1). See e.g., Perie, J., et al., 1990, Id.

### III. Methods of Use

In one aspect, there is provided a method of treating a disease or disorder in a subject in need thereof. The method includes administering to the subject a therapeutically effective amount of a compound as disclosed herein, a microparticle composition as disclosed herein, a nanoparticle composition as disclosed herein, or a pharmaceutical composition as disclosed herein.

In one aspect, there is provided a method of treating a disease or disorder in a subject in need thereof. The method includes administering to the subject a therapeutically effective amount of a compound as disclosed herein, a microparticle composition as disclosed herein, or a pharmaceutical composition as disclosed herein.

In one aspect, there is provided a method of treating a disease or disorder in a subject in need thereof. The method includes administering to the subject a therapeutically effective amount of a compound as disclosed herein, a nanoparticle composition as disclosed herein, or a pharmaceutical composition as disclosed herein.

In one aspect, there is provided a method of treating cancer or a viral infection in a subject in need thereof. The method includes administering to the subject a therapeutically effective amount of a compound as disclosed herein, a microparticle composition as disclosed herein, a nanoparticle composition as disclosed herein, or a pharmaceutical composition as disclosed herein.

In one aspect, there is provided a method of treating cancer or a viral infection in a subject in need thereof. The method includes administering to the subject a therapeutically effective amount of a compound as disclosed herein, a microparticle composition as disclosed herein, or a pharmaceutical composition as disclosed herein.

In one aspect, there is provided a method of treating cancer or a viral infection in a subject in need thereof. The method includes administering to the subject a therapeutically effective amount of a compound as disclosed herein, a nanoparticle composition as disclosed herein, or a pharmaceutical composition as disclosed herein.

Further to any aspect of treatment disclosed herein, in some embodiments, the pharmaceutical composition is an oral pharmaceutical composition or an intravenous pharmaceutical composition. In some embodiments, the pharmaceutical composition is an oral pharmaceutical composition. In some embodiments, the pharmaceutical composition is an intravenous pharmaceutical composition.

In some embodiments, the administration is oral administration or intravenous administration. In some embodiments, the administration is oral administration. In some embodiments, the administration is intravenous administration.

Further to any aspect of treatment of cancer, in some embodiments the cancer is cancer of the ovary, lung including non-small cell lung cancer, breast or pancreas. In some embodiments, the cancer is ovarian cancer. In some embodiments, the cancer is lung cancer. In some embodiments, the cancer is non-small cell lung cancer. In some embodiments, the cancer is breast cancer. In some embodiments, the cancer is pancreatic cancer.

Further to any aspect of treatment of a viral infection, in some embodiments the viral infection is influenza, hepatitis B, hepatitis C, cytomegalovirus, a herpes infection including those caused by varicella zoster, herpes simplex type 1, herpes simplex type 2, herpes simplex type 6 and herpes simplex type 8, Epstein-Barr virus, retroviral infection including those caused by SIV, HIV-1 and HIV-2, ebola virus, adenovirus and papilloma virus.

### IV. Pharmaceutical Compositions

In another aspect, the present invention provides a pharmaceutical composition or pharmaceutical formulation including compounds disclosed herein in combination with a pharmaceutically acceptable excipient (e.g., carrier).

A “pharmaceutically acceptable carrier,” as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

Accordingly, in another aspect, there is provided a pharmaceutical composition including a compound with structure of Formula (I)

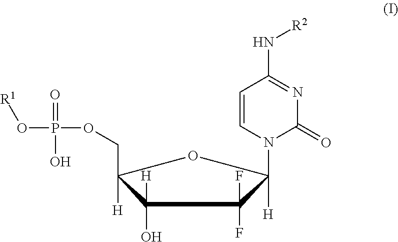

or pharmaceutically acceptable salt thereof, in combination with a pharmaceutically acceptable excipient. In this compound, R¹ is hydrogen, unsubstituted C₁₂-C₂₄ alkyl, or substituted or unsubstituted C₁₂-C₂₇ heteroalkyl. R² is hydrogen or —CO—R³. R³ is substituted or unsubstituted C₁-C₂₄ alkyl.

In some embodiments, R¹ is hydrogen.

In some embodiments, R¹ is unsubstituted C₁₂-C₂₄ alkyl, e.g., C₁₂, C₁₃, C₁₄, C₁₅, C₁₆, C₁₇, C₁₈, C₁₉, C₂₀, C₂₁, C₂₂, C₂₃, or C₂₄, preferably C₁₈.

In some embodiments, R² is hydrogen. In some embodiments, R² is —CO—R³. In some embodiments, R³ is unsubstituted C₁-C₂₄ alkyl, e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, or C₂₄.

In some embodiments, the pharmaceutical composition includes a compound having the structure of any one of Formulae (Ia), (Ib), (Ic) or (Id) following:

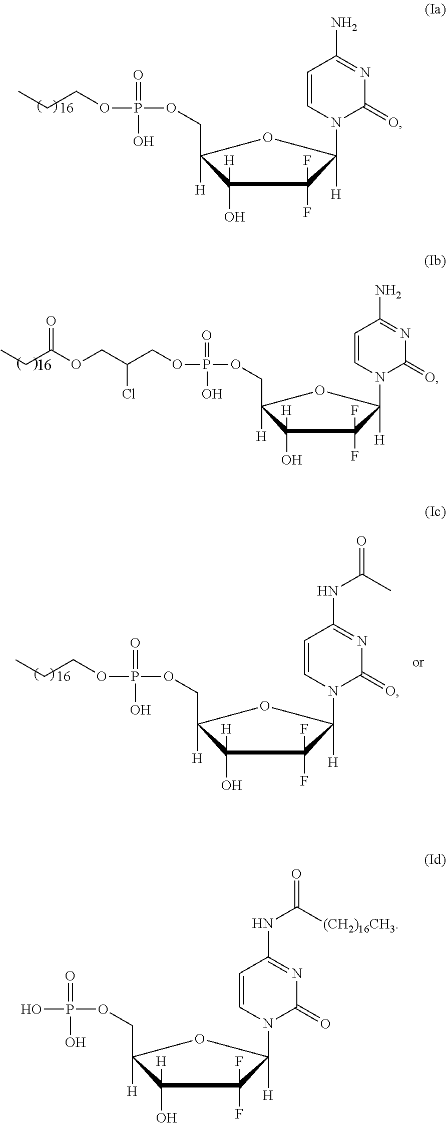

In some embodiments of the pharmaceutical composition, the compound with structure of Formula (I) is present as a microparticle composition as disclosed herein. In some embodiments, the microparticle composition includes a compound with structure of any of Formulae (Ia), (Ib), (Ic) or (Id).

In some embodiments of the pharmaceutical composition, the compound with structure of Formula (I) is present as a nanoparticle composition as disclosed herein. In some embodiments, the nanoparticle composition includes a compound with structure of any of Formulae (Ia), (Ib), (Ic) or (Id).

Further to any aspect disclosed herein, in some embodiments the pharmaceutical composition includes optical isomers, diastereomers, or pharmaceutically acceptable salts of the compounds disclosed herein.

The compounds of the invention can be administered alone or can be co-administered to a subject. Co-administration is meant to include simultaneous or sequential administration of the compounds individually or in combination (more than one compound). The preparations can also be combined, when desired, with other active substances (e.g. to reduce metabolic degradation).

A. Formulations

The compounds of the present invention can be prepared and administered in a wide variety of oral, parenteral, and topical dosage forms. Thus, the compounds of the present invention can be administered by injection (e.g. intravenously, intramuscularly, intracutaneously, subcutaneously, intraduodenally, or intraperitoneally). Also, the compounds described herein can be administered by inhalation, for example, intranasally. Additionally, the compounds of the present invention can be administered transdermally. It is also envisioned that multiple routes of administration (e.g., intramuscular, oral, transdermal) can be used to administer the compounds of the invention. Accordingly, the present invention also provides pharmaceutical compositions comprising a pharmaceutically acceptable carrier or excipient and one or more compounds of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the compounds of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compounds of the invention can also be incorporated into liposomes or administered via transdermal pumps or patches. Pharmaceutical admixtures suitable for use in the present invention include those described, for example, in PHARMACEUTICAL SCIENCES (17th Ed., Mack Pub. Co., Easton, Pa.) and WO 96/05309, the teachings of both of which are hereby incorporated by reference and for all purposes.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the present invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

B. Effective Dosages

Pharmaceutical compositions provided by the present invention include compositions wherein the active ingredient is contained in a therapeutically effective amount. An “effective amount” of a compound or composition is an amount sufficient to accomplish a stated purpose relative to the absence of the compound or composition, e.g., to achieve the effect for which it is administered. An example of an effective amount is an amount sufficient to contribute to the treatment, prevention, or reduction of a symptom or symptoms of a disease, which is referred to as a “therapeutically effective amount.” A “reduction” of a symptom or symptoms (and grammatical equivalents of this phrase) means decreasing of the severity or frequency of the symptom(s), or elimination of the symptom(s). The actual amount effective for a particular application will depend, inter alia, on the condition being treated. For example, when administered in methods to treat cancer, such compositions will contain an amount of active ingredient effective to achieve the desired result (e.g. decreasing the number of cancer cells in a subject).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to transcriptional inhibition); presence of other diseases or other health-related problems; kind of concurrent treatment; and complications from any disease or treatment regimen. Other therapeutic regimens or agents can be used in conjunction with the methods and compounds of the invention. The exact amounts will depend on the purpose of the treatment, and will be ascertainable by one skilled in the art using known techniques (see, e.g., Lieberman, PHARMACEUTICAL DOSAGE FORMS (vols. 1-3, 1992); Lloyd, THE ART, SCIENCE AND TECHNOLOGY OF PHARMACEUTICAL COMPOUNDING (1999); Pickar, Dosage Calculations (1999); and Remington: THE SCIENCE AND PRACTICE OF PHARMACY, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of decreasing physiologic activity (e.g., enzymatic activity) as measured, for example, using the methods described.

Therapeutically effective amounts for use in humans may be determined from animal models. For example, a dose for humans can be formulated to achieve a concentration that has been found to be effective in animals. The dosage in humans can be adjusted by monitoring inhibition and adjusting the dosage upwards or downwards, as described above.

Dosages may be varied depending upon the requirements of the patient and the compound being employed. The dose administered to a patient, in the context of the present invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

C. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

### V. Examples

Statistical analyses were completed using ANOVA followed by Fisher's protected less significant procedure. A p value of ≦0.05 was considered significant.

### Example 1

### Analytical Chemistry

Proton NMR spectra were recorded on a 300 MHz Varian UNITYplus™ or a 500 MHz Varian INOVA. Chemical shifts (δ) of ¹H NMR spectra were recorded in parts per million (ppm) relative to tetramethylsilane (TMS), which was the reference (δ=0 ppm). ¹H NMR data are reported according to the following order: chemical shift, integration (i.e., number of hydrogen atoms), multiplicity (s=singlet, d=doublet, t=triplet, q=quartet, m=multiplet, br=broad, brs=broad singlet), and coupling constant in Hertz (Hz). High resolution mass spectra were acquired in electrospray positive and negative ionization modes by direct injection onto an IonSpec 9.4T QFT-FTMS system in the mass spectrometry facility of the Department of Chemistry and Biochemistry at the University of Texas at Austin. The concentrations of dCyd and dUrd in the dCDA assay were determined using an Agilent 1260 Infinity high performance liquid chromatography (HPLC) with an Agilent ZORBAX® Eclipse Plus C18 column (4.6×150 mm, 5 μm) attached to a ZORBAX® Eclipse Plus guard column (Agilent Technologies, Inc., Santa Clara, Calif.).

All commercially available chemical reagents were purchased from Sigma-Aldrich (St. Louis, Mo.) or Thermo Fisher Scientific Inc. (Pittsburgh, Pa.) and were used as received unless noted. Gemcitabine hydrochloride (HCl) was from U.S. Pharmacopeia (Rockville, Md.). Soy lecithin was from Alfa Aesar (Ward Hill, Mass.). HOAt was from CreoSalus, Inc. (Louisville, Ky.). Water was purified using a Millipore Milli-Q® Advantage A10 (Billerica, Mass.). Air or moisture-sensitive reactions were performed under an atmosphere of argon. Thin-layer chromatography (TLC) on Whatman™ silica gel plates (UV₂₅₄) from Fisher Scientific was used to monitor the reaction progress. Silica gel—grade 60 (230-400 mesh) from Fisher Scientific was used for column chromatography to purify reaction products. The chemical structures of final compounds were confirmed using NMR and high resolution mass spectrometry. Purities of compounds 4, 8, 9, 12, and 13 were ≧95.0% based on NMR data.

### Example 2

### Synthesis of 4-N-stearoyl gemcitabine (4)

### 3′,5′-O-Bis(tert-Butoxycarbonyl)gemcitabine (2)

Gemcitabine HCl salt (1) (200 mg, 0.67 mmol) in 13.3 mL of 1N potassium hydroxide (KOH) was cooled to 4° C. To this solution, di-tert-butyl dicarbonate (Boc₂O, 1.483 g, 6.8 mmol) in about 13.3 mL of anhydrous dioxane was added over 10 min under argon atmosphere. The reaction mixture was stirred at 22° C. for 1 h and extracted with EtOAc. The organic layer was washed with brine, dried over anhydrous sodium sulfate (Na₂SO₄) and filtered. Solvent was removed under reduced pressure. The residue was added to Boc₂O (1.483 g, 6.8 mmol) in 13.3 mL of anhydrous dioxane and 13.3 mL of 1N KOH at 20° C. The reaction progress was monitored by TLC. After 1 h, the reaction mixture was extracted to EtOAc. The organic layer was washed with brine, dried over anhydrous Na₂SO₄, and filtered. Solvent was removed, and the crude product was purified by column chromatography (DCM:acetone, 1:1). The desired product fractions were pooled and dried to yield 219 mg of 2 (yield of 71%). ¹H NMR (500 MHz, acetone-d⁶) δ 7.60 (1H, d, J=7.6 Hz, 6-CH), 6.34 (1H, brs, 1′-CH), 5.97 (1H, d, J=7.6 Hz, 5-CH), 5.29 (1H, brs, 3′-CH), 4.53-4.39 (3H, m, 4′-CH, 5′A-CH, 5′B-CH), 2.82 (2H, s, NH₂) 1.50, 1.47 (18H, two s, (CH₃)₃C).

### 4-N-heptadecylcarbonyl-3′,5′-O-Bis(tert-butoxycarbonyl) gemcitabine (3)

A solution of 2 (219 mg, 0.47 mmol), stearic acid (149 mg, 0.52 mmol) and HOAt (70 mg, 0.52 mmol) in anhydrous DCM was pre-cooled to 4° C., and EDCI (109 mg, 0.57 mmol) was added. The mixture was de-gassed by vacuum sonication and then stirred at room temperature under argon for about 40 h. Water (15 mL) was added to the reaction mixture and extracted with the mixture of EtOAc and hexane (2:1). The combined organic phase was washed with saturated ammonium chloride (NH₄Cl) and brine and then dried over anhydrous Na₂SO₄. The solvent was evaporated, and the residue was purified by column chromatography (EtOAc:Hexane, 3:7). The conjugated amide 3 was isolated as a white powder (319 mg, 92%). ¹H NMR (300 MHz, acetone-d⁶) δ 9.90 (1H, s, NHCO), 8.03 (1H, d, J=7.8 Hz, 6-CH), 7.45 (1H, d, J=7.5 Hz, 5-CH), 6.38 (1H, t, J=8.7 Hz, 1′-CH), 5.40-5.30 (1H, m, 3′-CH), 4.56-4.44 (3H, m, 4′-CH and 5′-CH₂), 2.57 (2H, t, J=7.5 Hz, CO—CH₂), 1.71-1.65 (2H, m, CO—CH₂—CH₂), 1.50, 1.47 (18H, two s, (CH₃)₃C), 1.40-1.20 (28H, m, CH₂), 0.90-0.87 (3H, m, terminal CH₃).

### 4-N-stearoyl gemcitabine (4)

To a stirred solution of the compound 3 (319 mg, 0.44 mmol) in 7 mL of DCM, about 1.5 ml of TFA was added. This solution was stirred at room temperature for 2 h, and excess TFA was removed under reduced pressure. The concentrated sample was co-distilled with DCM for 5 times. The crude sample was chromatographed on silica gel (DCM:ethanol, 94:6). See e.g., Immordino, M. L., et al., 2004, Id. The desired fractions were pooled, and the solvent was evaporated to yield 4 as a white powder (162 mg, 70%). ¹H NMR (500 MHz, pyridine-d⁵) δ 11.97 (1H, s, NHCO), 8.75 (1H, d, J=7.6 Hz, 6-CH), 7.74 (1H, d, J=7.6 Hz, 5-CH), 6.99 (1H, t, J=7.2 Hz, 1′-CH), 5.18-5.11 (1H, m, 3′-CH), 4.47-4.28 (3H, overlapping m, 4′-CH and 5′-CH₂), 2.67 (2H, t, J=7.4 Hz, CO—CH₂), 1.83-1.76 (2H, m, CO—CH₂—CH₂), 1.34-1.20 (28H, m, CH₂), 0.87 (3H, t, J=6.9 Hz, terminal CH₃). ESI-HRMS [M+H]⁺ m/z calculated for C₂₇H₄₆F₂N₃O₅: 530.3406, found: 530.3401.

### Example 3

### Synthesis of 2′-2′-difluoro-2′-deoxycytidine-5′-octadecylphosphate (8)

### 3′-O-(tert-butoxycarbonyl)-2′-2′-difluoro-cytidine (5A)

The mixture of gemcitabine HCl (200 mg, 0.67 mmol) and Na₂CO₃ (354 mg, 3.3 mmol) in about 3.3 mL of water and 13.3 mL of dioxane was added to Boc₂O (147 mg, 0.67 mmol). The mixture was stirred at room temperature for 48 h. After 15 mL of water was added, the mixture was extracted with EtOAc (3×50 mL). The combined organic extracts were washed with brine, dried over anhydrous Na₂SO₄, and concentrated under reduced pressure. The crude sample was chromatographed on silica gel (DCM:acetone, 1:2). The desired fractions were pooled, and the solvent was evaporated to yield 212 mg of 5A (87%). ¹H NMR (300 MHz, acetone-d⁶) δ 7.75 (1H, d, J=7.5 Hz, 6-CH), 6.34 (1H, t, J=9.1 Hz, 1′-CH), 6.04 (1H, d, J=7.8 Hz, 5-CH), 5.40-5.31 (1H, m, 3′-CH), 4.23-4.19 (1H, m, 4′-CH), 4.00-3.82 (2H, overlapping m, 5′A-CH, 5′B-CH), 1.51 (9H, s, (CH₃)₃C).

### 4-N-3′-O-Bis(tert-butoxycarbonyl)-2′-2′-difluoro-cytidine (5B)

Boc₂O (1.26 g, 5.8 mmol) was added to a stirred solution of 5A (212 mg, 0.58 mmol) in 10 mL of dioxane. The resultant mixture was maintained at 37° C. in a rotary shaker at 250 rpm for 3 days. Water (25 mL) was added to the sample, and the mixture was extracted with EtOAc (3×50 mL). The organic layer was washed with brine and dried over anhydrous Na₂SO₄. The concentrated sample was chromatographed on silica gel (EtOAc:hexanes, 1:1). The desired fractions were pooled, and the solvent was evaporated to yield 196 mg of 5B (73%). ¹H NMR (300 MHz, acetone-d⁶) δ 8.25 (1H, d, J=7.5 Hz, 6-CH), 7.30 (1H, d, J=7.5 Hz, 5-CH), 6.36 (1H, t, J=8.6 Hz, 1′-CH), 5.37-5.28 (1H, m, 3′-CH), 4.34-4.28 (1H, m, 4′-CH), 4.08-3.98 (1H, m, 5′A-CH), 3.87 (1H, m, 5′B-CH), 1.52, 1.50 (18H, two s, (CH₃)₃C).

### Octadecylphosphate (6)

Octadecanol (5 g, 18.48 mmol) and triethylamine (4.8 g, 47.52 mmol) were partially dissolved in 50 mL of DCM under argon. POCl₃ (2.8 g, 18.48 mmol) was added drop-wise and heated to reflux for 2 h. See e.g., Bligh, E. G. & Dyer, W. J., 1959, Id.; Perie, J., et al., 1990, Id. The reaction mixture was filtered to remove triethylamine hydrochloride, and the filtrate was added to 0.2 N NaHCO₃ (370 mL). After 15 h stirring at room temperature, 370 mL of acetone was added, and the white precipitate was recovered by filtration. The precipitate was washed with acetone and re-dissolved in 400 mL of water. Another 260 mL of acetone was added, and the precipitate was recovered, washed with acetone, dissolved in a homogeneous mixture of 200 mL of CHCl₃, 400 mL of CH₃OH, and 200 mL of 0.1 N HCl, and stirred for 1 h at room temperature. A mixture of 200 mL of CHCl₃ and 200 mL of water was added, and the organic layer was isolated. The aqueous phase was extracted with CHCl₃ (2×100 mL). The combined organic layer was evaporated to dryness and lyophilized (2.2 g, 34%). ¹H NMR (300 MHz, CDCl₃:CD₃OD, 4:1) δ 3.98 (2H, q, J=6.8 Hz, CH₂OP), 1.70-1.62 (2H, m, CH₂CH₂OP), 1.39-1.19 (30H, m, CH₂ from C18 chain), 0.88 (3H, t, J=6.8 Hz, terminal CH₃). ESI-HRMS [M−H]⁻ m/z calculated for C₁₈H₃₈O₄P⁻: 349.2513, found: 349.2515.

### 4-N-3′-O-Bis(tert-butoxycarbonyl)-2′-2′-difluoro-cytidine-5′-octadecylphosphate (7)

The powders of compound 5 (100 mg, 0.22 mmol) and octadecylphosphate (220 mg, 0.62 mmol) were mixed and lyophilized for 15 h. To the lyophilized powder, TPS (154 mg, 0.5 mmol) and 2 mL of pyridine were added under argon environment, and the reaction was stirred at 38-40° C. for 24 h. A few drops of water were added, and solvent was removed under reduced pressure. The crude oil was chromatographed on silica gel, eluting first with CHCl₃:CH₃OH (24:1) and then with CHCl₃:CH₃OH (9:1). The fractions of the desired product (R_{f}=0.2 in CHCl₃:CH₃OH, 9:1) were pooled, and the solvent was evaporated to dryness. Compound 7 was isolated as a white powder (156 mg, 89%). ¹H NMR (300 MHz, CDCl₃:CD₃OD, 4:1) δ 7.94 (1H, d, J=7.2 Hz, 6-CH), 7.30 (1H, d, J=7.5 Hz, 5-CH), 6.35 (1H, t, J=8.6 Hz, 1′-CH), 5.29-5.18 (1H, m, 3′-CH), 4.29-4.10 (3H, overlapping m, 4′-CH, 5′A-CH, 5′B-CH), 3.89-3.78 (2H, m, CH₂OP), 1.60 (2H, t, J=6.6 Hz, CH₂CH₂OP), 1.52, 1.50 (18H, two s, (CH₃)₃C), 1.39-1.19 (30H, m, CH₂ from C18 chain), 0.88 (3H, t, J=6.6 Hz, terminal CH₃). ESI-HRMS [M−H]⁻ m/z calculated for C₃₇H₆₃F₂N₃O₁₁P⁻: 794.4174, found: 794.4162.

### 2′-2′-difluoro-2′-deoxycytidine-5′-octadecylphosphate (8)

To a stirred solution of 7 (95 mg, 0.12 mmol) in 6 mL of DCM, about 0.9 ml of TFA was added. This solution was stirred at room temperature for 2 h. Excess TFA was removed under reduced pressure, and the concentrated sample was co-distilled with DCM for 5 times. The crude sample was column-purified on silica gel by eluting with 20%, 40%, and 50% CH₃OH in CHCl₃, sequentially. The desired fractions with the R_{f} value of 0.25 (CHCl₃:CH₃OH, 3:2) were pooled and evaporated to dryness to yield 41 mg of 8 (58%). ¹H NMR (300 MHz, CDCl₃:CD₃OD, 4:1) δ 7.88 (1H, d, J=8.1 Hz, 6-CH), 6.17 (1H, t, J=6.9 Hz, 1′-CH), 6.06 (1H, d, J=7.2 Hz, 5-CH), 4.42-4.00 (4H, overlapping m, 3′-CH, 4′-CH, 5′A-CH, 5′B-CH), 3.94-3.71 (2H, m, CH₂OP), 1.62 (2H, t, J=6.6 Hz, CH₂CH₂OP), 1.39-1.19 (30H, m, CH₂ from C18 chain), 0.88 (3H, t, J=6.6 Hz, terminal CH₃). ESI-HRMS [M−H]⁻ m/z calculated for C₂₇H₄₇F₂N₃O₇P⁻: 594.3125, found: 594.3125.

### Example 4

### Synthesis of 4-N-acetyl-2′-2′-difluoro-2′-deoxycytidine-5′-octadecylphosphate (9)

### 4-N-acetyl-2′-2′-difluoro-2′-deoxycytidine-5′-octadecylphosphate (9)

Acetic anhydride (150 μL) and 8 (16.5 mg, 0.03 mmol) in 2 mL of CH₃OH was refluxed for 15 h, and the resultant mixture was co-distilled with CH₃OH five times. The resultant sample was vacuum dried overnight to obtain 14 mg of 9 (73%). ¹H NMR (300 MHz, CDCl₃:CD₃OD, 4:1) δ 7.90-8.10 (1H, m, 6-CH), 6.30-6.05 (2H, two m, 1′-CH, 5-CH), 4.42-3.80 (6H, overlapping m, 3′-CH, 4′-CH, 5′A-CH, 5′B-CH, CH₂OP), 2.04 (3H, s, NHCOCH₃) 1.65-1.50 (2H, m, CH₂CH₂OP), 1.39-1.19 (30H, m, CH₂ from C18 chain), 0.88 (3H, t, J=6.3 Hz, terminal CH₃). ESI-HRMS [M−H]⁻ m/z calculated for C₂₉H₄₉F₂N₃O₈P⁻: 636.3231, found: 636.3220.

### Example 5

### Synthesis of 2′-2′-difluoro-2′-deoxycytidine 5′(3-stearoyloxy-2-rac-chloropropyl)phosphate (12)

### 1-Stearate-2-hydroxy-3-phosphatidic Acid (10)

Glycerol monostearate (1 g, 2.79 mmol) and triethylamine (0.726 g, 7.17 mmol) were partially dissolved in 12 mL of DCM under argon. POCl₃ (0.428 g, 2.79 mmol) was added drop-wise and heated to reflux for 2 h. See e.g., Bligh, E. G. & Dyer, W. J., 1959, Id.; Perie, J., et al., 1990, Id. The reaction mixture was filtered to remove triethylamine hydrochloride, and the filtrate was added to 0.2 N NaHCO₃ (56 mL). After 15 h of stirring at room temperature, 56 mL of acetone was added, and the white precipitate was recovered by filtration. The precipitate was washed with acetone and re-dissolved in 60 mL of water. Another 40 mL of acetone was added, and the precipitate was recovered. It was then washed with acetone, dissolved in a homogeneous mixture of 100 mL of CHCl₃, 200 mL of CH₃OH, and 100 mL of 0.1 N HCl, and stirred for 1 h at room temperature. A mixture of 100 mL of CHCl₃ and 100 mL of water was added, and the organic layer was isolated. The aqueous phase was extracted with CHCl₃ (2×50 mL). The combined organic layer was evaporated to dryness and lyophilized to obtain 10 (720 mg, 59%). ¹H NMR (300 MHz, CDCl₃:CD₃OD, 4:1) δ 4.10-4.03 (2H, m, COOCH₂), 4.00-3.90 (3H, overlapping m, CH₂OP, CHOH), 2.27 (2H, t, J=7.4 Hz, COCH₂), 1.60-1.48 (2H, m, COCH₂CH₂), 1.30-1.10 (28H, m, CH₂ from C18 chain), 0.80 (3H, t, J=6.0 Hz, terminal CH₃). ESI-HRMS [M−H]⁻ m/z calculated for C₂₁H₄₂O₇P⁻: 437.2674, found: 437.2673.

### 4-N-3′-O-Bis(tert-butoxycarbonyl)-2′-2′-difluoro-2′-deoxycytidine 5′(3-stearoyloxy-2-rac-chloropropyl)phosphate (11)

The powders of 5 (50 mg, 0.11 mmol) and 10 (110 mg, 0.25 mmol) were mixed and lyophilized for 15 h. To the lyophilized powder, TPS (74 mg, 0.25 mmol) and 1 mL of pyridine were added under argon environment, and the reaction was stirred at 38-40° C. for 24 h. A few drops of water were added, and the solvent was removed under reduced pressure. The crude oil was chromatographed on silica gel, eluting first with CHCl₃:CH₃OH (24:1) and then with CHCl₃:CH₃OH (9:1). The fractions of desired product were pooled, and the solvent was evaporated to dryness. The compound 11 was isolated as a white powder (56 mg, 62%). ¹H NMR (300 MHz, CDCl₃:CD₃OD, 4:1) δ 7.95 (1H, d, J=7.8 Hz, 6-CH), 7.32 (1H, d, J=6.9 Hz, 5-CH), 6.34 (1H, t, J=7.6 Hz, 1′-CH), 5.18-5.31 (1H, m, 3′-CH), 4.60-4.10 (7H, overlapping m, 4′-CH, 5′A-CH, 5′B-CH, COCH₂, CH₂OP), 3.82-3.71 (1H, m, CHCl), 2.39-2.29 (2H, m, COCH₂), 1.68-1.56 (2H, m, CH₂CH₂CO), 1.53, 1.51 (18H, two s, (CH₃)₃C), 1.38-1.19 (28H, m, CH₂ from C18 chain), 0.88 (3H, t, J=6.6 Hz, terminal CH₃). ESI-HRMS [M−H]⁻ m/z calculated for C₄₀H₆₆ClF₂N₃O₁₃P⁻: 900.3995, found: 900.3997, [M+H]⁺ m/z calculated for C₄₀H₆₈ClF₂N₃O₁₃P⁻: 902.4141, found: 902.4150.

### 2′-2′-difluoro-2′-deoxycytidine 5′(3-stearoyloxy-2-rac-chloropropyl)phosphate (12)

To a stirred solution of 11 (50 mg, 0.055 mmol) in 4 mL of DCM, about 0.5 ml of TFA was added. This solution was stirred at room temperature for 2 h. The excess TFA was removed under reduced pressure, and the concentrated sample was co-distilled with DCM for 5 times. The crude sample was column-purified on silica gel by eluting with 5%, 10%, and 15% CH₃OH in CHCl₃, sequentially. The desired fractions with the R_{f} value of 0.2 (CH₃OH:CHCl₃, 2:1) were pooled and evaporated to dryness to yield 18 mg of compound 12 (47%). ¹H NMR (500 MHz, CDCl₃:CD₃OD, 4:1) δ 7.78 (1H, d, J=7.6 Hz, 6-CH), 6.22 (1H, t, J=6.9 Hz, 1′-CH), 5.95-5.92 (1H, m, 5-CH), 4.24-3.66 (9H, overlapping m, 3′-CH, 4′-CH, 5′A-CH, 5′B-CH, COCH₂, CH₂OP, CHCl), 2.36-2.29 (2H, m, COCH₂), 1.62-1.58 (2H, m, CH₂CH₂CO), 1.31-1.19 (28H, m, CH₂ from C18 chain), 0.88 (3H, t, J=7.0 Hz, terminal CH₃). ESI-HRMS [M−H]⁻ m/z calculated for C₂₀H₅₀ClF₂N₃O₉P⁻: 700.2947, found: 700.2958, [M+H]⁺ m/z calculated for C₃₀H₅₂ClF₂N₃O₉P⁺: 702.3092, found: 702.3089.

### Example 6

### Synthesis of 4-N-octadecanoyl-2′-2′-difluoro-2′-deoxycytidine 5′-sodiumphosphate (13)

### 4-N-Octadecanoyl-2′-2′-difluoro-2′-deoxycytidine 5′-sodiumphosphate (13)

Compound 4 (50 mg, 0.094 mmol) and triethylamine (0.072 g, 0.72 mmol) were partially dissolved in 2 mL of DCM under argon. POCl₃ (0.0329 g, 0.21 mmol) was added drop-wise and heated to reflux for 2 h. The reaction mixture was filtered to remove triethylamine hydrochloride, and the filtrate was added to 0.2 N NaHCO₃ (6 mL). After 15 h stirring at room temperature, 6 mL of acetone was added, and the white precipitate was recovered by filtration. The precipitate was washed with acetone and re-dissolved in 6 mL of water. Acetone (4 mL) was added, and the precipitate was recovered. The precipitate was washed with acetone several times and dried under vacuum to obtain 7 mg of pale yellow powder of 13 (12% yields). ¹H NMR (300 MHz, CD₃OD) δ 8.4-8.3 (1H, m, 6-CH), 7.6-7.5 (1H, m, 5-CH), 6.3-6.2 (1H, m, 1′-CH), 4.2-3.8 (4H, overlapping m, 3′-CH, 4′-CH and 5′-CH₂), 2.44 (2H, m, CO—CH₂), 1.8-1.7 (2H, m, CO—CH₂—CH₂), 1.34-1.20 (28H, m, CH₂), 0.89 (3H, m, terminal CH₃). ESI-HRMS [M−H]⁻ m/z calculated for C₂₇H₄₅F₂N₃O₈P⁺: 608.2918, found: 608.2933, [M+H]⁺ m/z calculated for C₂₇H₄₇F₂N₃O₈P⁺: 610.3063, found: 610.3064.

### Example 7

### Cell Lines and Cell Culture

Human leukemia cell line CCRF-CEM (CCL-119), human pancreatic cancer cell lines PANC-1 (CRL-1469), MIA PaCa-2 (CRL-1420), and BxPC-3 (CRL-1687), human breast adenocarcinoma cell line MCF-7 (HTB-22), and mouse lung cancer cell line TC-1 (CRL-2785) were from the American Type Culture Collection (Rockville, Md.). CCRF-CEM-AraC-8C cells (hENT1 deficient) and CCRF-CEM/dCK^{-/-} cells (dCK deficient) were kindly provided by Dr. Buddy Ullmann (Oregon Health & Science University, Portland, Oreg.) and Dr. Margaret Black (Washington State University, Pullman, Wash.), respectively. L1210 wt and L1210 10K were kindly supplied by Dr. Lars Petter Jordheim (Université Claude Bernard Lyon I, Lyon, France). TC-1-GR cells were previously developed in our lab. CCRF-CEM, CCRF-CEM-AraC-8C, CCRF-CEM/dCK^{-/-}, L1210 wt, L1210 10K, TC-1, and TC-1-GR cells were cultured in RPMI 1640 medium. MCF-7 and PANC-1 cells were cultured in Dulbecco's modified Eagle medium (DMEM), MIA PaCa-2 cells were cultured in DMEM medium with 2.5% horse serum. All media were supplemented with 10% fetal bovine serum (FBS), 100 U/mL of penicillin, and 100 μg/mL of streptomycin (all from Invitrogen, Carlsbad, Calif.).

### Example 8

### Preparation of Nanoparticles

Gemcitabine derivative-containing nanoparticles were prepared as previously described. See e.g., Sloat, B. R., et al., 2011, Id.; Sloat, B. R., et al., 2010, Id. Briefly, 3.5 mg of soy lecithin, 0.5 mg of glycerol monostearate, and 1 mg of gemcitabine derivative were placed into a 7 mL glass vial. One mL of de-ionized and filtered (0.22 μm) water was added into the mixture, which was then maintained on a 90-105° C. hot plate while stirring until the formation of homogenous slurry. Tween™ 20 was added in a step wise manner to a final concentration of 1% (v/v). The resultant emulsions were allowed to cool to room temperature while stirring to form nanoparticles. Particle size and zeta potential were determined using a Malvern Zetasizer Nano ZS (Westborough, Mass.).

### Example 9

### In Vitro Cytotoxicity Assay

Cells (5,000/well) were seeded in 96-well plates. After overnight incubation, they were treated with various concentrations of gemcitabine HCl, gemcitabine derivatives, or gemcitabine derivatives in nanoparticles at 37° C., 5% CO₂. TC-1 and TC-1-GR cells were incubated for 48 h. CCRF-CEM, CCRF-CEM-AraC-8C, CCRF-CEM/dCK^{-/-}, L1210 wt, L1210 10K, and MCF-7 cells were incubated for 72 h, and MIA PaCa-2 and PANC-1 for 96 h. Gemcitabine HCl was dissolved in phosphate-buffered saline (PBS), and gemcitabine derivatives were dissolved in dimethyl sulfoxide (DMSO). The maximum amount of DMSO added per well was 1 μL, which was found non-toxic. Compound 13 was not used in the in vitro cytotoxicity assay because it was not sufficiently solubilized in DMSO. The number of viable cells after the incubation was determined using an MTT assay. Briefly, 3-(4,5-dimethylthiazol-2-yl)-2,5-diphenyltetrazolium bromide (20 μl, 5 mg/mL) was added in each well and incubated for 3 h. Formazan crystals were solubilized with acidified isopropanol (150 μl) (for CCRF-CEM, CCRF-CEM-AraC-8C, CCRF-CEM/dCK^{-/-}, L1210 wt, and L1210 10K cells) or DMSO (150 μl) (for TC-1, TC-1-GR, MCF-7, PANC-1, and MIA PaCa-2). Absorbance was measured using a BioTek Synergy™ HT Multi-Mode Microplate Reader (BioTek Instruments, Winooski, Vt.) at 570 nm and 630 nm. The fraction of affected (dead) cells (Fa) and the fraction of unaffected (live) cells (Fu) at every dose were calculated, and the Log(Fa/Fu) values were plotted against the Log(concentration of gemcitabine). IC₅₀ was the dose at Log(Fa/Fu)=0, See e.g., Chou, T. C. & Talalay, P., 1984, Adv. Enzyme Regul. 22:27-55. The experiment was repeated at least three times.

### Example 10

### Partial Purification dCDA and dCDA Activity Assay

dCDA was partially purified from BxPC-3 cells as previously described. The pellet of 1×10⁸ cells was suspended in 4 mL of 20 mM Tris buffer (pH 7.5) containing 5 mM potassium chloride (KCl), 1 mM dithiothreitol, 40 μl of streptomycin sulfate (12.74 mg/mL), and 50 μl of protease inhibitor cocktail. See e.g., Bergman, A. M., et al., 2004, Id.; Laliberté, J., et al., 1994, Id. The suspended cells were sonicated and centrifuged for 30 min at 20,000 g. Ammonium sulfate was added to reach 40% saturation, stirred for 1 h, and centrifuged at 36,000 g for 20 min at 4° C. Ammonium sulfate was added to the supernatant to reach 55% saturation, mixed for 1 h and centrifuged at 36,000 g for 20 min at 4° C. The pellet was resuspended in 1 mL of 20 mM Tris buffer (pH 7.5) and desalted by overnight dialysis against water. Protein concentration was measured using Bradford reagent from Sigma-Aldrich. The dCDA activity assay was carried out as described previously with slight modifications. See e.g., Bergman, A. M., et al., 2004, Id.; Ruiz van Haperen, V. W., et al., 1993, Id. Briefly, 55 μl of partially purified dCDA (3.2 mg/mL) and 0.5 mM dCyd (20 μl) in a total volume of 200 μl of 20 mM of Tris buffer (pH 7.5) were incubated at 37° C. for 15 min. The reaction was terminated by the addition of 50 μl of trichloroacetic acid (40%, w/v) and chilling on ice for 20 min. Protein was precipitated by centrifugation at 10,000 g for 10 min, and the supernatant was neutralized with 500 μl of trioctylamine and 1,1,2-trichloro-trifluoroethane (1:4). The mixture was centrifuged at 10,000 g for 1 min, and the upper layer was analyzed using HPLC (detection wavelength, 260 nm; mobile phase, 10% methanol in water). The relevant peaks were quantified to determine the concentrations of dCyd and dUrd. For the competition assay, 20 μl of gemcitabine HCl or gemcitabine derivative-containing nanoparticles, with molar equivalent concentrations of gemcitabine derivatives, were included in the reaction mixture. Controls include a reaction with substrate (dCyd) but without an inhibitor and a reaction without substrate and inhibitors, but with blank nanoparticles (i.e., gemcitabine derivative-free).

### Example 11

### Solid Lipid Nanoparticles

GemC18 and other lipophilic monophosphorylated gemcitabine derivatives, 8, 9, 12, and 13, were incorporated into solid lipid nanoparticles prepared from lecithin/glycerol monostearate-in-water emulsions as described previously to prepare GemC18-NPs, 8-NPs, 9-NPs, 12-NPs, and 13-NPs, respectively. See e.g., Sloat, B. R., et al., 2011, Id.; Sloat, B. R., et al., 2010, J. Control. Release 141(1):93-100. The sizes of the resultant nanoparticles were 150-175 nm, with a polydispersity index of 0.2-0.3. The zeta potentials of the nanoparticles were −27 mV to −40 mV. See Table S1 following, wherein the data are shown as mean±S.D. (n≧3). “S.D.” refers to standard deviation, as customary in the art. To evaluate the extent to which the gemcitabine derivatives and their corresponding nanoparticles can overcome various mechanisms of gemcitabine resistance, the cytotoxicities of them in cancer cells that are dCK deficient, hENT1 deficient, or over-expressing RRM1 or RRM2 were determined. In addition, the ability of selected gemcitabine derivative-containing nanoparticles to competitively inhibit the deamination activity of partially purified dCDA was evaluated and compared to that of gemcitabine HCl as well.

TABLE S1
Size and Zeta Potential of the Gemcitabine Derivative-Containing
Nanoparticles.

| | Particle Size | Polydispersity | Zeta Potential |
|---|---|---|---|
| | (nm) | Index | (mV) |
| Blank NPs | 155 ± 7 | 0.24 ± 0.01 | −32.6 ± 0.6 |
| GemC18-NPs | 150 ± 16 | 0.24 ± 0.04 | −30.2 ± 2.0 |
| 8-NPs | 149 ± 24 | 0.35 ± 0.03 | −40.5 ± 1.4 |
| 9-NPs | 147 ± 32 | 0.30 ± 0.05 | −27.5 ± 1.0 |
| 12-NPs | 176 ± 5 | 0.32 ± 0.06 | −40.5 ± 3.1 |
| 13-NPs | 168 ± 37 | 0.30 ± 0.04 | −32.3 ± 1.9 |

### Example 12

### Lipophilic Gemcitabine Derivatives and their Nanoparticles can Overcome Deoxycytine Kinase Deficiency

The in vitro cytotoxicities of the gemcitabine derivatives and their nanoparticles in human leukemia cell line, CCRF-CEM, and its derivative line, CCRF-CEM/dCK^{-/-}, were evaluated and compared to that of gemcitabine HCl. The IC₅₀ value of gemcitabine HCl in the parent CCRF-CEM cells was 2.9±1.8 nM (Table 1), which was 6-77-fold smaller than that of the gemcitabine derivatives, GemC18, 8, 9, 12, and the derivatives in nanoparticles, GemC18-NPs, 8-NPs, 9-NPs, 12-NPs, and 13-NPs (Table 1), demonstrating that in CCRF-CEM cells, gemcitabine HCl was more cytotoxic than the gemcitabine derivatives, alone or in nanoparticles. Overall, this finding is in agreement with data from our previous studies, which showed that the GemC18-NPs were less cytotoxic than gemcitabine HCl in various cancer cells including the CCRF-CEM, likely because the gemcitabine needs to be hydrolyzed from the GemC18 or GemC18-NPs to be effective. See e.g., Sloat, B. R., et al., 2011, Id. In fact, doubling the incubation time of the GemC18-NPs with the TC-1 lung cancer cells enabled the GemC18-NPs to kill the same proportion of the cancer cells as gemcitabine HCl. Finally, it appears that the IC₅₀ values of GemC18, 8, 9, 12 were not significantly different from that of their corresponding nanoparticles in CCRF-CEM cells (Table 1), indicating that the incorporation of the gemcitabine derivatives into nanoparticles did not improve their cytotoxicities against the CCRF-CEM cells.

TABLE 1
The IC₅₀ Values of Gemcitabine, Gemcitabine Derivatives,
and the Derivatives in Nanoparticles in CCRF-CEM and
CCRF-CEM/dCK^{-/-} Cells.

| | | CCRF- | Ratio of IC₅₀ in |
|---|---|---|---|
| | CCRF-CEM | CEM/dCK^{-/-} | CCRF-CEM/dCK^{-/-} |
| | (nM) | (μM) | to that in CCRF-CEM |
| Gemcitabine | 2.9 ± 1.8 | 240.4 ± 29.0 | 82,897 |
| HCl | | | |
| GemC18 | 19.4 ± 13.3 | 47.3 ± 8.7 | 2,438 |
| 8 | 195.9 ± 28.1 | 38.3 ± 1.2 | 196 |
| 9 | 58.1 ± 18.5 | 62.3 ± 5.3 | 1,072 |
| 12 | 49.8 ± 5.9 | 87.4 ± 10.1 | 1,755 |
| GemC18-NPs | 16.3 ± 4.5 | 13.3 ± 2.6 | 816 |
| 8-NPs | 152.1 ± 15.7 | 3.8 ± 0.2 | 25 |
| 9-NPs | 86.5 ± 9.4 | 5.9 ± 1.4 | 68 |
| 12-NPs | 58.8 ± 15.6 | 2.8 ± 0.2 | 48 |
| 13-NPs | 222.2 ± 68.7 | 11.3 ± 3.5 | 51 |

In the CCRF-CEM/dCK^{-/-} cells, the IC₅₀ value of gemcitabine HCl was 240.4±29.0 μM, which was 82,897-fold greater than that in the parent CCRF-CEM cells (Table 1). In contrast, the IC₅₀ values of GemC18, 8, 9, 12, GemC18-NPs, 8-NPs, 9-NPs, 12-NPs, and 13-NPs in the CCRF-CEM/dCK^{-/-} cells were only 25- to 2,438-fold greater than their IC₅₀ values in the parent CCRF-CEM cells (Table 1). In the dCK deficient CCRF-CEM-dCK^{-/-} cells, the IC₅₀ values of the gemcitabine derivatives and their corresponding nanoparticles were 3-86-fold smaller than that of gemcitabine HCl. In other words, the gemcitabine derivatives, alone or in nanoparticles, were more cytotoxic to the CCRF-CEM/dCK^{-/-} cells than gemcitabine HCl. Therefore, it appears that the gemcitabine derivatives and their nanoparticles are less dependent on dCK to be active than gemcitabine HCl. The finding with the gemcitabine derivatives in nanoparticles is new, and the finding with the derivatives alone is consistent with previous data generated in dCK over-expressing or dCK deficient cells using other phospholipid gemcitabine derivatives and gemcitabine phosphoramidate. See e.g., Alexander, R. L., et al., 2005, Cancer Chemother. Pharmacol. 56(1):15-21; Wu, W., et al., 2007, J. Med. Chem. 50(15):3743-3746. Moreover, the ratios of the IC₅₀ value of the same compound in the CCRF-CEM/dCK^{-/-} to that in the parent CCRF-CEM cells seem to show that the monophosphorylated gemcitabine derivatives (i.e., 8, 9, and 12) were less dependent on the dCK to be active than GemC18, which is not monophosphorylated (Table 1). Importantly, it appears that the incorporation of the gemcitabine derivatives into nanoparticles rendered the monophosphorylated gemcitabine derivatives further less dependent on the dCK to be active (Table 1). For example, for the gemcitabine derivatives alone, the ratio of the IC₅₀ value of CCRF-CEM/dCK^{-/-} to CCRF-CEM was 2,438 for the GemC18, 196-1,755 for the other monophosphorylated derivatives (Table 1). However, for the gemcitabine derivatives in nanoparticles, the ratio was 816 for the GemC18-NPs, but only 25-68 for 8-NPs, 9-NPs, 12-NPs, and 13-NPs (Table 1). Gemcitabine is phosphorylated by dCK, and the monophosphorylation of gemcitabine is the rate limiting step in the activation of gemcitabine. See e.g., Mini, E., et al., 2006, Id.; Ueno, H., et al., 2007, Id. Therefore, it was expected that the monophosphorylated gemcitabine derivatives are less dependent on dCK to be active than the GemC18. Interestingly, it appears that the combination of monophosphorylation of gemcitabine and the incorporation of the lipophilic monophosphorylated gemcitabine derivative into nanoparticles can more effectively bypass the rate limiting step of phosphorylation in gemcitabine activation.

To further validate this finding, the cytotoxicities of gemcitabine HCl and selected gemcitabine derivatives in nanoparticles were evaluated in another dCK deficient cell line, the murine leukemia cells L1210 10K. The IC₅₀ value of gemcitabine HCl in the parent L1210 wt cells was 1.3±0.3 nM, which was 17,046-fold smaller than the IC₅₀ value of gemcitabine HCl in the dCK deficient L1210 10K cells (22.2±3.7 μM) (Table 2). Interestingly, in the L1210 10K cells, GemC18-NPs and 8-NPs were 4- and 8-fold more cytotoxic than gemcitabine HCl, respectively (Table 2). In addition, the IC₅₀ values of GemC18-NPs and 8-NPs in the L1210 10K cells were only 17-431-fold greater than that in the L1210 wt cells (Table 2), further confirming that the incorporation of the gemcitabine derivatives in nanoparticles makes them less dependent on dCK to be active. We did not investigate the mechanism of hydrolysis of the monophosphorylated gemcitabine derivatives, but it is likely that they were hydrolyzed in between the lipophilic chain and the phosphate group, similar to the hydrolysis of 1-β-D-arabinofuranosylcytosine (ara-C) or gemcitabine phospholipid derivatives. See e.g., Alexander, R. L., et al., 2005, Id.; Raetz, C. R., et al., 1977, Science 196(4287):303-305.

TABLE 2
The IC₅₀ Values of Gemcitabine HCl, GemC18-NPs, and 8-NPs in
L1210 wt and L1210 10K Cells.

| | | | Ratio of IC50 | Ratio of IC50 |
|---|---|---|---|---|
| | | | values in | in L1210 10K |
| | L1210 wt | L1210 10K | L1210 10K | to that in |
| | (nM) | (μM) | cells* | L1210 wt |
| Gemcitabine | 1.3 ± 0.3 | 22.2 ± 3.7 | 1 | 17,046 |
| HCl | | | | |
| GemC18-NPs | 13.1 ± 0.3 | 5.6 ± 0.1 | 4 | 431 |
| 8-NPs | 172.5 ± 55.2 | 2.9 ± 0.3 | 8 | 17 |

*Ratio is the IC₅₀ values of gemcitabine HCl divided by that of the nanoparticles.

### Example 13

### Lipophilic Gemcitabine Derivatives and their Nanoparticles can Overcome Gemcitabine Resistance Related to Ribonucleotide Reductase MI Over-Expression

RRM1 plays a substantial role in DNA synthesis and gemcitabine resistance. See e.g., Bergman, A. M., et al., 2005, Cancer Res. 65(20):9510-9516; Ceppi, P., et al., 2006, Ann. Oncol. 17(12):1818-1825; Davidson, J. D., et al., 2004, Cancer Res. 64(11):3761-3766; Goan, Y.-G., et al., 1999, Cancer Res. 59(17):4204-4207; Ohtaka, K., et al., 2008, Oncol. Rep. 20:279-286; Rosell, R., et al., 2004, Clin. Cancer Res. 10(4):1318-1325; Yen, Y., 2003, Clin. Cancer Res. 9(12):4304-4308. Previously, we developed a tumor cell line that over-expresses RRM1 (TC-1-GR). In TC-1-GR cells, GemC18-NPs were significantly more toxic than gemcitabine HCl, although in the parent TC-1 cells, GemC18-NPs were significantly less toxic than gemcitabine HCl. Importantly, in mice with pre-established TC-1-GR tumors, GemC18-NPs significantly inhibited the tumor growth, but gemcitabine HCl did not show any significant anti-tumor activity. In the present study, the IC₅₀ values of the new lipophilic monophosphorylated gemcitabine derivatives and their nanoparticles in both TC-1 and TC-1-GR cells were determined to evaluate their ability to overcome gemcitabine resistance caused by RRM1 over-expression. As expected, in TC-1 cells, gemcitabine HCl was more cytotoxic (IC₅₀, 14.7±2.8 nM) than the gemcitabine derivatives and their nanoparticles (Table 3). However, in TC-1-GR cells, the majority of gemcitabine derivatives (except 12) and all the gemcitabine derivatives in nanoparticles were more cytotoxic than gemcitabine HCl (2- to 10-fold) (Table 3). Importantly, in TC-1-GR cells, the IC₅₀ value of gemcitabine HCl was 36.7±5.1 μM, which was 2,497-fold greater than that in TC-1 cells. In contrast, the IC₅₀ values of GemC18, 8, 9, 12 and the nanoparticles, GemC18-NPs, 8-NPs, 9-NPs, 12-NPs, and 13-NPs, in TC-1-GR cells were only 23- to 177-fold greater than that in TC-1 cells (Table 3), demonstrating that, the gemcitabine derivatives and their nanoparticles are less sensitive to gemcitabine resistance caused by RRM1-over-expression than gemcitabine HCl. Incorporation of the gemcitabine derivatives into nanoparticles tended to make the derivatives, particularly the GemC18, more cytotoxic to the RRM1-over-expressing TC-1-GR cells. However, unlike what was observed in the dCK deficient cells (Tables 1, 2), it seemed that in RRM1-over-expressing TC-1-GR cells, monophosphorylation of gemcitabine did not add any additional benefits compare with GemC18.

TABLE 3
The IC₅₀ Values of Gemcitabine HCl, Gemcitabine Derivatives,
and the Derivatives in Nanoparticles in TC-1 and TC-1-GR Cells.

| | | | Ratio of IC₅₀ | Ratio of |
|---|---|---|---|---|
| | | | values in | IC₅₀ in |
| | TC-1 | TC-1-GR | TC-1-GR | TC-1-GR to |
| | (nM) | (μM) | cells* | that in TC-1 |
| Gemcitabine | 14.7 ± 2.8 | 36.7 ± 5.1 | 1 | 2,497 |
| HCl | | | | |
| GemC18 | 132.1 ± 17.2 | 7.7 ± 2.4 | 5 | 58 |
| 8 | 245.5 ± 39.9 | 21.1 ± 1.7 | 2 | 86 |
| 9 | 210.7 ± 85.0 | 9.3 ± 2.5 | 4 | 44 |
| 12 | 430.0 ± 52.4 | 76.3 ± 10.5 | 0.5 | 177 |
| GemC18-NPs | 59.8 ± 18.4 | 3.6 ± 0.2 | 10 | 60 |
| 8-NPs | 371.3 ± 27.3 | 8.4 ± 0.5 | 4 | 23 |
| 9-NPs | 258.6 ± 60.9 | 10.1 ± 0.9 | 4 | 39 |
| 12-NPs | 405.7 ± 114.1 | 10.5 ± 2.0 | 3 | 26 |
| 13-NPs | 395.5 ± 40.7 | 9.0 ± 2.6 | 4 | 23 |

*Ratio is the IC₅₀ values of gemcitabine HCl divided by that of the derivatives or derivatives in nanoparticles.

### Example 14

### Cytotoxicities of Gemcitabine Derivatives and their Nanoparticles in Cancer Cells Over-Expressing Different Levels of RRM2

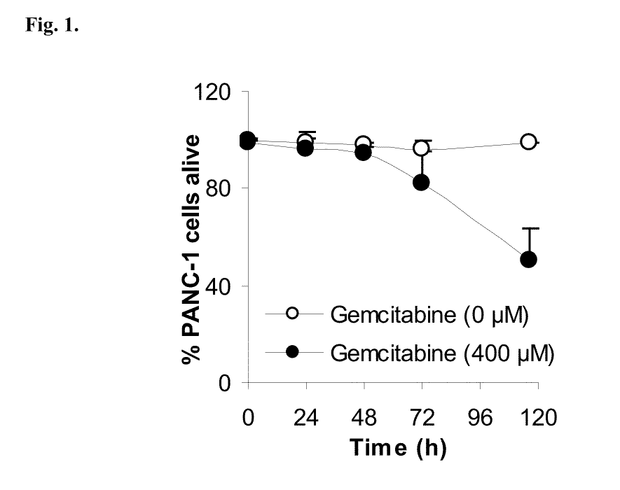

It was reported that MIA PaCa-2 and PANC-1 cells both over-expressed RRM2, but PANC-1 cells express ˜70% more RRM2 than MIA PaCa-2. See e.g., Duxbury, M. S., et al., 2003, Oncogene 23(8):1539-1548. In MIA PaCa-2 cells, the IC₅₀ value of gemcitabine HCl and GemC18 NPs were 49.7±17.7 nM and 40.6±8.2 nM, respectively (Table 4), all other gemcitabine derivatives and their nanoparticles were less toxic than gemcitabine HCl (Table 4). However, more than 50% of PANC-1 cells were still alive after 96 h of incubation with 400 μM of gemcitabine HCl (Table 4 and FIG. 1). Data from a trypan blue exclusion assay showed that it took 116 h to kill 50% of PANC-1 cells with 400 μM of gemcitabine HCl (FIG. 1). The IC₅₀ values of the gemcitabine derivatives and their nanoparticles in PANC-1 cells were 5.8 to 58.7 μM (Table 4). The IC₅₀ values of gemcitabine HCl in PANC-1 cells was more than 8,000-fold greater than that in the MIA PaCa-2 cells, but the IC₅₀ values of gemcitabine derivatives and their nanoparticles in PANC-1 cells were only 24- to 239-fold greater than those in MIA PaCa-2 cells (Table 4). In other words, the gemcitabine derivatives and their nanoparticles were less sensitive than gemcitabine HCl to gemcitabine resistance caused by RRM2 over-expression. Again, monophosphorylation of gemcitabine did not add any additional benefits in its cytotoxicity against the RRM2-over-expressing PANC-1 cells, and except for the GemC18-NPs, a conclusion that the incorporation of gemcitabine derivatives into nanoparticles makes them more cytotoxic cannot be drawn. Previously, Duxbury et al. reported that the IC₅₀ values of gemcitabine in MIA PaCa-2 cells and PANC-1 cells were 40 nM and 50 nM, respectively. The IC₅₀ value of gemcitabine HCl in MIA PaCa-2 cells determined in the present study is comparable to what was reported by Duxbury et al., but the PANC-1 cells were significantly more resistant to gemcitabine HCl in our study. See e.g., Duxbury, M. S., et al., 2003, Oncogene 23(8):1539-1548.

TABLE 4
The IC₅₀ Values of Gemcitabine HCl, Gemcitabine Derivatives, and
the Derivatives in Nanoparticles in MIA PaCa-2 and PANC-1 Cells.

| | | | Ratio of IC₅₀ in |
|---|---|---|---|
| | MIA PaCa-2 | PANC-1 | PANC-1 to that |
| | (nM) | (μM) | in MIA PaCa-2 |
| Gemcitabine | 49.7 ± 17.7 | >400 | >8,000 |
| HCl | | | |
| GemC18 | 133.0 ± 60.4 | 6.0 ± 1.1 | 45 |
| 8 | 835.9 ± 163.8 | 50.4 ± 4.3 | 60 |
| 9 | 204.6 ± 39.5 | 38.5 ± 5.7 | 188 |
| 12 | 245.1 ± 38.0 | 58.7 ± 14.2 | 239 |
| GemC18-NPs | 40.6 ± 8.2 | 5.8 ± 0.6 | 143 |
| 8-NPs | 201.7 ± 50.2 | 6.2 ± 1.5 | 31 |
| 9-NPs | 290.0 ± 84.6 | 8.8 ± 1.8 | 30 |
| 12-NPs | 380.0 ± 98.3 | 22.0 ± 3.5 | 58 |
| 13-NPs | 357.6 ± 172.2 | 8.7 ± 2.9 | 24 |

### Example 15

### Cytotoxicities of Gemcitabine Derivatives and their Nanoparticles in Nucleoside Transporter Deficient Cancer Cells

It is known that nucleoside transporters are a prerequisite for the cellular uptake of gemcitabine. See e.g., Damaraju, V. L., et al., 2003, Oncogene 22(47):7524-7536. Therefore, in the hENT1 deficient CCRF-CEM-AraC-8C cells, the IC₅₀ value of the gemcitabine HCl was 998.8±9.4 nM, 344 times greater than that in the parent CCRF-CEM cells (Table 5). However, the IC₅₀ values of gemcitabine derivatives and their nanoparticles in CCRF-CEM-AraC-8C cells were only 4-fold to 35-fold greater than that in the parent CCRF-CEM cells (Table 5), indicating that the gemcitabine derivatives, alone or in nanoparticles, are less sensitive to hENT1 deficient than gemcitabine HCl, possibly because the gemcitabine derivatives can diffuse into cells without the help of the nucleoside transporters, and the gemcitabine derivatives in nanoparticles can be taken up by cells via endocytosis. However, the cytotoxicities of the monophosphorylated gemcitabine derivatives and their nanoparticles in the CCRF-CEM-AraC-8C cells are not significantly different from that of gemcitabine HCl (Table 5). Only the GemC18 and GemC18-NPs were 2.7- and 12.3-fold more cytotoxic than gemcitabine HCl, respectively, in the hENT1-deficient CCRF-CEM-AraC-8C cells (Table 5), which was consistent with our previous data. It is likely that the phosphate group on the gemcitabine derivatives made them less effective in entering the hENT1 deficient cells.

TABLE 5
The IC₅₀ Values of Gemcitabine HCl, Gemcitabine Derivatives,
and the Derivatives in Nanoparticles in CCRF-CEM and
CCRF-CEM-AraC-8C Cells.

| | | | Ratio of | Ratio of IC₅₀ in |
|---|---|---|---|---|
| | | | IC₅₀ values | CCRF-CEM- |
| | | | in CEM- | AraC-8C |
| | CCRF-CEM | CCRF-CEM- | AraC-8C | to that in |
| | (nM) | AraC-8C (nM) | cells* | CCRF-CEM |
| Gemcitabine | 2.9 ± 1.8 | 998.8 ± 9.4 | 1 | 333 |
| HCl | | | | |
| GemC18 | 19.4 ± 13.3 | 369.4 ± 235.1 | 2.7 | 19 |
| 8 | 195.9 ± 28.1 | 806.5 ± 111.8 | 1.2 | 4 |
| 9 | 58.1 ± 18.5 | 575.3 ± 97.1 | 1.7 | 10 |
| 12 | 49.8 ± 5.9 | 1766.9 ± 532.8 | 0.6 | 35 |
| GemC18-NPs | 16.3 ± 4.5 | 81.0 ± 17.9 | 12.3 | 5 |
| 8-NPs | 152.1 ± 15.7 | 942.6 ± 163.8 | 1.1 | 4 |
| 9-NPs | 86.5 ± 9.4 | 1325.1 ± 167.2 | 0.8 | 13 |
| 12-NPs | 58.8 ± 15.6 | 712.9 ± 342.9 | 1.4 | 12 |
| 13-NPs | 222.2 ± 68.7 | 1166.6 ± 293.1 | 0.9 | 5 |

*Ratio is the IC₅₀ values of gemcitabine HCl divided by that of the derivatives or derivatives in nanoparticles.

### Example 16

### Gemcitabine HCl, but not Gemcitabine Derivatives in Nanoparticles, Inhibits dCDA Activity

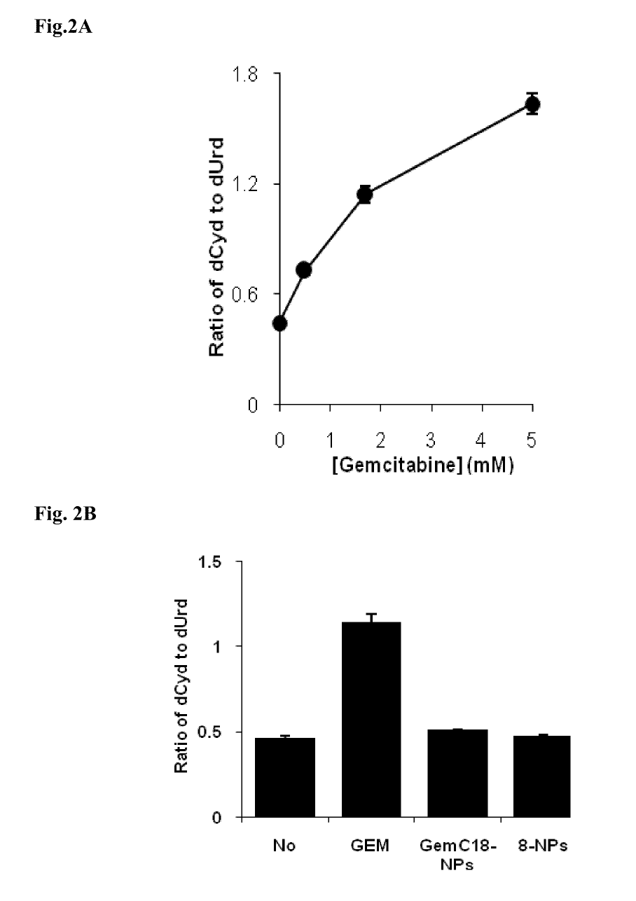

Previously, Bouffard et al. reported that gemcitabine HCl, as a substrate to dCDA, competitively inhibits the deamination of deoxycytidine (dCyd) by dCDA. See e.g., Bouffard, D. Y., et al., 1993, Id. In order to test whether the gemcitabine derivatives are still good substrates of dCDA and inhibit its activity, dCDA was partially purified from BxPC-3 human pancreatic cancer cells, and its deamination activity against dCyd was determined in the presence or absence of gemcitabine HCl or selected gemcitabine derivatives in nanoparticles. See e.g., Bergman, A. M., et al., 2004, Biochem. Pharmacol. 67(3):503-511; Laliberté, J. & Momparler, R. L., 1994, Cancer Res. 54(20):5401-5407; Ruiz van Haperen, V. W., et al., 1993, Eur. J. Cancer 29A (No. 15):2132-2137. The nanoparticles, but not the gemcitabine derivatives alone, were used due to the poor water solubility of the gemcitabine derivatives. GemC18-NPs and 8-NPs were chosen because of their greater cytotoxicity in previous in vitro cytotoxicity assays. Deoxyuridine (dUrd) was not detected in the control reaction (with dCyd, but no dCDA) indicating that any observed dUrd would be due to the deamination of dCyd by dCDA. As shown in FIG. 2A, dCyd was converted to dUrd in the presence of the partially purified dCDA. Gemcitabine HCl competitively inhibited the conversion of dCyd to dUrd, and the extent of the inhibition was increased by increasing the concentration of gemcitabine HCl (FIG. 2A). However, GemC18-NPs and 8-NPs did not significantly inhibit the deamination activity of dCDA (FIG. 2B), confirming that gemcitabine HCl, but not GemC18-NPs and 8-NPs, can competitively inhibit the deamination of dCyd by dCDA. Therefore, the gemcitabine derivatives in nanoparticles are no longer good substrates of dCDA, and it is expected that they can potentially overcome gemcitabine resistance caused by deamination. This finding is in agreement with data from previous studies, showing that other gemcitabine derivatives were no longer good substrates of dCDA as well. See e.g., Song, X., et al., 2005, Mol. Pharmaceutics. 2(2):157-167; Bergman, A. M., et al., 2004, Id.

### Example 17

### Oral 5′-O-Stearyl Phosphate Gemcitabine Nanoparticles Inhibited Tumor Growth in Mice

Introduction:

In previous studies, we have shown that the 4-N-stearoyl gemcitabine nanoparticles (GemC18-NPs) developed in our lab were significantly more effective than gemcitabine HCl in controlling tumor growth in mouse models, when given intravenously or orally. In the present study, we evaluated the anti-tumor activity of one of the novel gemcitabine derivatives we synthesized, 5′-O— stearyl phosphate gemcitabine (compound 8), when incorporated into similar solid lipid nanoparticles (8-NPs) and given orally to mice.

Methods:

8-NPs were prepared as reported previously. See e.g., Sloat, B. R., et al., 2011, Id. Briefly, 3.5 mg of soy lecithin (Alfa Aesar; Ward Hill, Mass.), 0.5 mg of glycerol monostearate (Gattefosse Corp; Paramus, N.J.), PEG2000 (11.6% w/w, Avanti Polar Lipids Inc; Alabaster, Ala.), and 2.5 mg of 8 were placed into a 7 mL scintillation glass vial. One mL of de-ionized (Millipore Milli-Q®; Billerica, Mass.) and filtered (0.22 μm) water was added into the mixture, which was then maintained on a 90-95° C. hot plate while stirring, with occasional water-bath sonication (Bransonic® Ultrasonic Cleaner, Danbury, Conn.), until the formation of homogenous slurry. Tween™ 20 (Sigma; St. Louis, Mo.) was added in a step wise manner to a final concentration of 1% (v/v). The resultant emulsions were allowed to cool to room temperature while stirring to form nanoparticles. Animal protocol was approved by the IACUC at the University of Texas at Austin. Female C57BL/6 mice (18-20 g, 6-8 weeks, n=5) were subcutaneously (s.c.) injected with mouse TC-1 lung cancer cells (ATCC # CRL-2785, 5×105 cells/mouse) in the right flank. Mouse hair was carefully trimmed at the injection site 1 day prior to the injection. Treatment with 8-NPs, 8 dissolved in vegetable oil (8-in-oil, ConAgra Foods; Omaha, Nebr.), or sterile mannitol (5% w/v) were started on day 7, and mice were orally gavaged every other day until the endpoint. One group of mice was injected via the tail vein with the 8-NPs, twice a week. The dose of 8 was 250 μg per mouse per dose. Tumor size was measured every other day, and tumor volume was calculated as: volume (mm³)=[length×(width)]/2.

Results and Conclusions:

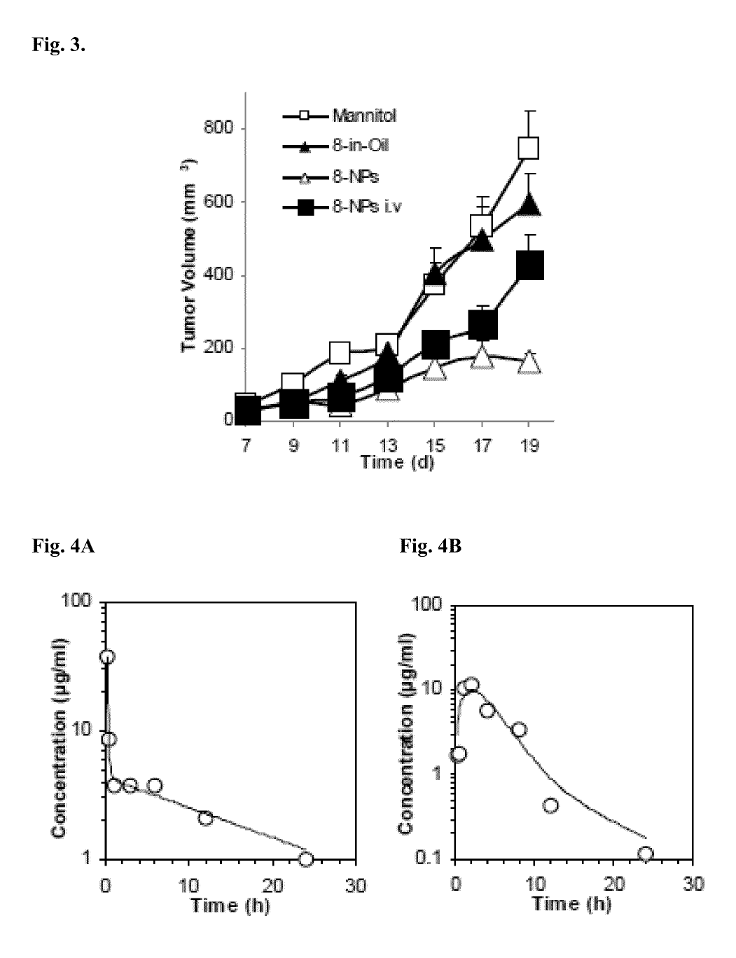

Oral 8-NPs significantly inhibited the growth of the TC-1 tumors in mice, whereas 8-in-vegetable oil did not show any significant anti-tumor activity (FIG. 3). Intravenous 8-NPs (8-NPs i.v.) were less effective than oral 8-NPs, likely because mice in the i.v. group were injected only twice a week, whereas mice in the oral group were gavaged every other day (FIG. 3). In conclusion, it appears that the 8-NPs, when given orally, showed a significant anti-tumor activity.

### Example 18

### GemC18-NPs Show a Relative Bioavailability of 70% when Given Orally to Mice

Introduction:

Previously, we have shown that our 4-(N)-stearoyl gemcitabine nanoparticles (GemC18-NPs) significantly inhibited the TC-1 mouse model lung cancer cells in a mouse model when given orally, whereas the GemC18-in-vegetable oil, at the same dose, was toxic to mice. In the present study, we evaluated the pharmacokinetics (PK) of GemC18 when the GemC18-NPs were administered orally (p.o.) or intravenously (i.v.) to mice.

Methods:

Healthy BALB/c mice (25-28 g, n=3) were dosed with GemC18-NPs with 1 mg of GemC18 by i.v. injection via the tail-vein or by oral gavage. The GemC18-NPs were prepared in 200 μL of sterile mannitol (5%, w/v) to achieve isotonicity. At predetermined time-points after intravenous or oral administration (i.e., 0.25, 0.5, 1, 2, 4, 6, 8, 12, 24, and/or 48 h), mice were euthanized, and blood was collected into heparinized tubes containing tetrahydrouridine (THU, 10 mg/mL) to further inhibit cytidine deaminase. Blood samples were centrifuged at 8000 rcf for 10 min to isolate plasma.

Due to the lack of an internal standard for GemC18, a hydrolysis method was used to detect the concentration of GemC18 in plasma (Pasut et al, 2007). Briefly, 75 μL of plasma was collected, to which 25 μL of AraU solution (10 mg/mL) was added as an internal standard. To the mixture, 100 μL of 2 N NaOH was added, and the final mixture was vortexed and incubated at 40° C. for 1 h to hydrolyze GemC18 into gemcitabine. Acetonitrile (800 μL) and H₃PO₄ (75 mL, 1.4 M) were added after the incubation, followed by centrifugation at 13,000 rpm for 10 min. The supernatant was collected and dried under vacuum. The remaining residue was dissolved in 100 μL of PBS (2.5 mM) and centrifuged at 13,000 rpm for 10 min. The supernatant was collected and subjected to HPLC to determine the concentration of gemcitabine. The mobile phase was 5 mM sodium acetate (pH 6.0) and methanol (95/5, v/v). The detection wavelength for gemcitabine was 266 nm. Standard curve was constructed using gemcitabine hydrochloride.

The following PK parameters were derived by fitting the data into two compartmental models using PKSolver (Zhang et al, 2010) or WinNonlin: maximum plasma concentration (Cₘₐₓ), time to reach Cmax (tₘₐₓ), area under the plasma concentration-time curve from time 0 to t hours (AUC₀₋ₜ), AUC from time zero to infinity (AUC_{0-inf}), area under the mean plasma concentration-time curve from time 0 to t hours (AUMC₀₋ₜ), area under the mean plasma concentration-time curve from time 0 to infinity (AUMC_{0-inf}), mean residence time from time 0 to t hours (MRT₀₋ₜ), and terminal half life (t_{1/2}).

Results and Conclusions:

The plasma gemcitabine concentration at different time points after the i.v. and oral administration of the GemC18-NPs is shown in FIGS. 4A-4B, respectively. Shown in Table 6 are the PK parameters after i.v. or oral administrations. The relative oral bioavailability was determined to be around 70%. Data in Table 6 were derived after the gemcitabine concentration was converted to 4-(N)-stearoyl gemcitabine concentration.

TABLE 6
PK parameters after GemC18-NPs were given to mice

| Intravenous injection | | | Oral gavage | | |
|---|---|---|---|---|---|
| Parameter | Unit | Observed | Parameter | Unit | Observed |
| k10 | 1/h | 2.21 | Tmax | h | 2.02 |
| k12 | 1/h | 5.62 | Cmax | μg/ml | 17.56 |
| k21 | 1/h | 0.19 | t1/2Alpha | h | 1.39 |
| t1/2Alpha | h | 0.09 | t1/2Beta | h | 6.84 |
| t1/2Beta | h | 13.02 | t1/2Ka | h | 1.29 |
| C0 | μg/ml | 434.34 | AUC0-t | μg/ml * h | 109.22 |
| V | (mg)/ | 0.002 | AUC0-inf | μg/ml * h | 112.31 |
| | (μg/ml) | | | | |
| CL | (mg)/ | 0.004 | AUMC | [symbol] | 659.78 |
| | (μg/ml)/h | | | | |
| V2 | (mg)/ | 0.06 | MRT | h | 5.87 |
| | (μg/ml) | | | | |
| CL2 | (mg)/ | 0.01 | V/F | (mg)/ | 0.02 |
| | (μg/ml)/h | | | (μg/ml) | |
| AUC0-t | μg/ml * h | 156.66 | CL/F | (mg)/ | 0.0146677 |
| | | | | (μg/ml)/h | |
| AUC0-inf | μg/ml * h | 196.47 | V2/F | (mg)/ | 0.01 |
| | | | | (μg/ml) | |
| AUMC | [symbol] | 2690.01 | CL2/F | (mg)/ | 0.00015 |
| MRT | h | 13.69 | | (μg/ml)/h | |

### Example 19

### A Gemcitabine Derivative Carried by PLGA Nanoparticles Shows Strong Anti-Tumor Activity

Active Pharmaceutical Ingredient:

4-(N)-stearoyl-gemcitabine (GemC18).

Drug Carrier/Excipients:

Poly(D,L lactic-co-glycolic acid) (PLGA), 75:25 Resomer®RG 752H (Sigma-Aldrich, St. Louis, Mo.), Lauric acid, 98% (Sigma-Aldrich), 1,2-dioleoyl-sn-glycero-3-phosphoethanolamine-N—[methoxy(polyethyleneglycol)-2000]ammonium salt) (PEG2000) (Avanti Polar Lipids, Inc., Alabaster, Ala.).

Formulation Methodology:

GemC18 was incorporated into PLGA nanoparticles through a solvent displacement method. The lipophilic phase contained 7.5 mg of PLGA and 1.35 mg Lauric acid. This lipid polymer/lipid mixture was dissolved in 0.75 ml tetrahydrofuran (THF) containing 0.45 mg of GemC18. The hydrophilic phase contained 1.85 mg PEG2000 and was dissolved in 3.75 mL of ethanol (4%, v/v). The lipophilic phase was added drop wise into the hydrophilic phase, and the resultant o/w-emulsions were agitated while stirring at 1000 rpm for 6 h to evaporate the THF and to allow the PLGA to precipitate to nanoparticles. The GemC18-loaded PLGA nanoparticles (PLGA-GemC18-NPs) were purified by centrifugation (14,000 rpm, 4° C., 15 min) and re-suspended in de-ionized and filtered (0.22 μm) water. As a control, GemC18-free, PLGA nanoparticles (PLGA-NPs) were also prepared as described above without the addition of GemC18.

Animal Study:

The anti-tumor activity of the PLGA-GemC18-NPs was evaluated in female C57BL/6 mice (18-20 g, 6-8 weeks, n=4-5) that were subcutaneously (s.c.) injected with TC-1 mouse lung cancer cells (ATCC # CRL-2785, 5×10⁵ cells/mouse) in the right flank. Mouse hair was carefully trimmed at the injection site 1 day prior to the injection. PLGA-GemC18-NPs, GemC18-free NP (PLGA-NPs), gemcitabine hydrochloride (HCl) (U.S. Pharmacopeia; Rockville, Md.) and sterile mannitol (5%, w/v) were intravenously (i.v) injected starting on day 11, twice a week until the end of the study. The dose of the GemC18 was 150 μg per mouse per dose, or the molar equivalent dose for gemcitabine HCl. Tumor size was determined 2-3 times a week and calculated using the formulas: diameter (mm)=[length+width]/2.

Results:

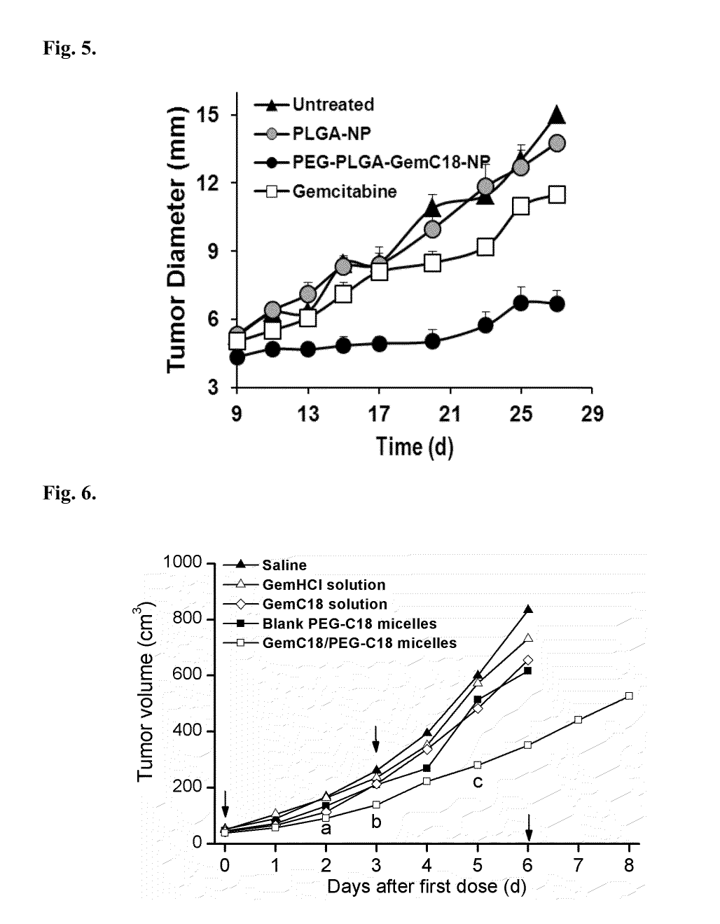

The particle size, polydispersity index, and zeta potential of the PLGA-GemC18-NPs were 210±9 nm, 0.17±0.03, −38±1 mV, respectively. As shown in FIG. 5, TC-1 tumors grew aggressively in mice that received sterile mannitol (as a negative control). The growth rate of the TC-1 tumors in mice that received the GemC18-free, PLGA-NPs was not different from that in mice that received the sterile mannitol, indicating that the PLGA nanoparticles per se were not responsible for any in vivo anti-tumor activity. Gemcitabine HCl only slightly delayed the TC-1 tumor growth, but the PLGA-GemC18-NPs significantly delayed the tumor growth.

Conclusions:

An intravenous formulation of GemC18-loaded PLGA nanoparticles was developed, and the PLGA-GemC18-NPs showed significant anti-tumor activity in a mouse model. The anti-tumor activity of the PLGA-GemC18-NPs was significantly stronger than that of the traditional gemcitabine HCl.

### Example 20

### Strong Anti-Tumor Activity from Stearoyl Gemcitabine Carried by a Novel Micelle

Micelle material (PEG-C18) was synthesized by conjugating hydrophilic methoxy-polyethylene glycol 2000 with a hydrophobic stearic acid derivative. Successful conjugation was confirmed by thin layer chromatography (TLC), proton nuclear magnetic resonance spectroscopy (¹H-NMR) and matrix-assisted laser desorption/ionization mass spectrometry (MALDI MS).

GemC18 was then incorporated into PEG-C18 micelles using a modified thin-film hydration method. Briefly, 0.25 mL of tetrahydrofuran containing 0.5 mg of GemC18 was dried under vacuum and then hydrated with 1 mL of PEG-C18 aqueous solution (10 mg/mL) under vigorous stirring in a 75° C. water bath. Micelles were obtained within 5 min and then cooled down to room temperature with constant water bath sonication. The resultant micelle preparation was filtrated through 0.2 μm filter and lyophilized to obtain a white solid, which was referred to as GemC18/PEG-C18 micelles. Characterization of GemC18/PEG-C18 micelles showed that the entrapment efficiency and drug loading percentage were 96.7±2.5% and 5.0±0.1%, respectively. The GemC18/PEG-C18 micelles had a spherical shape with a size of 43.1±3.8 nm in diameter.

The in vivo antitumor activity of GemC18/PEG-C18 micelles was evaluated in C57BL/6 mice with subcutaneously inoculated B16-F10 cells (5.0×10⁵/mouse) in the right flank. Treatments were given 6 days after tumor cell inoculation by tail vein injection with normal saline, Gemcitabine HCl (GemHCl) solution, GemC18 solution, blank PEG-C18 micelles, or GemC18/PEG-C18 micelles. The dose was 0.283 mg of GemHCl or molar equivalent amount of GemC18. All groups received a second dose three days after the first dose, while the GemC18/PEG-C18 micelle group also received a third dose 3 days after the second dose. Tumor sizes were measured with calipers in two perpendicular diameters every day and reported as tumor volume (V=½ [a×b²], a=longer diameter, b=shorter diameter).

As shown in FIG. 6, tumors in mice that were received normal saline grew uncontrolled. No significant difference in tumor volume was observed between blank PEG-C18 micelle groups and normal saline group, indicating that the blank micelles were pharmaceutically inert, and any therapeutic effect from the GemC18/PEG-C18 micelles should be attributed to GemC18 in the micelles. The sizes of the tumors in mice that were treated with GemHCl or GemC18 in solution were not significant different from that in mice that received normal saline at the end of treatment (Day 6, P=0.50 and 0.19 for GemHCl and GemC18, respectively). In contrast, GemC18/PEG-C18 micelles efficiently inhibited the tumor growth compared with normal saline, which was significant as early as on the second day after the first dose (P<0.05). Tumors in mice received GemC18/PEG-C18 micelles were significantly smaller than that in mice received GemHCl (P<0.05) from the third after the first dose, and on the fifth day after the first dose for the GemC18 in solution group (P<0.05). Finally, the body weights of mice that received various treatments were also recorded. A slight increase in body weight was observed at the end of treatment, but no significant difference was observed among the different groups of mice.

In conclusion, the PEG-C18 micelles significantly improved the antitumor activity of both GemC18 and Gemcitabine HCl, showing a great potential as a novel carrier of the lipophilic gemcitabine prodrug (GemC18).

As disclosed herein, novel lipophilic monophosphorylated gemcitabine derivatives were synthesized and incorporated into nanoparticles. All the gemcitabine derivatives and their nanoparticles showed significantly higher cytotoxicity than gemcitabine HCl in cells that are deficient in dCK, and the gemcitabine derivatives in nanoparticles were more cytotoxic than the corresponding gemcitabine derivatives. The majority of the gemcitabine derivatives and all the nanoparticles are also more cytotoxic than gemcitabine HCl to cancer cells that over-express RRM1 or RRM2. Finally, the gemcitabine derivatives in nanoparticles were no longer good substrates to dCDA and thus became resistance to deamination. Collectively, 5′-O-stearyl phosphate gemcitabine in nanoparticles showed the highest cytotoxicity to cells that are deficient in dCK, over-expressing RRM1, or over-expressing RRM2, and were resistant to deamination. Gemcitabine derivatives in nanoparticles showed anti-tumor activity when given orally or i.v. to tumor-bearing mice.

### VI. Embodiments

Embodiment 1. A compound with structure of Formula (I):

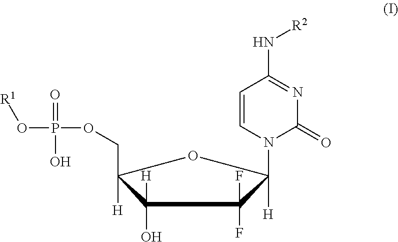

or pharmaceutically acceptable salt thereof, wherein R¹ is hydrogen, unsubstituted C₁₂-C₂₄ alkyl, or substituted or unsubstituted C₁₂-C₂₇ heteroalkyl; R² is hydrogen or —CO—R³; and R³ is substituted or unsubstituted C₁-C₂₄ alkyl.

Embodiment 2. The compound according to embodiment 1, wherein R² is hydrogen.

Embodiment 3. The compound according to any one of embodiments 1 to 2, wherein R¹ is unsubstituted C₁₂-C₂₄ alkyl.

Embodiment 4. The compound according to embodiment 3 with structure of Formula (Ia):

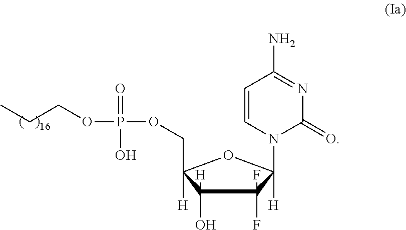

Embodiment 5. The compound according to any one of embodiments 1 to 2, wherein R¹ is substituted or unsubstituted C₁₂-C₂₇ heteroalkyl.

Embodiment 6. The compound according to embodiment 1, wherein R¹ is R⁴—CO—O-L¹-; R⁴ is substituted or unsubstituted C₁₀-C₂₀ alkyl; and L¹ is substituted or unsubstituted C₁-C₆ alkylene.

Embodiment 7. The compound according to embodiment 6 with structure of Formula (Ib):

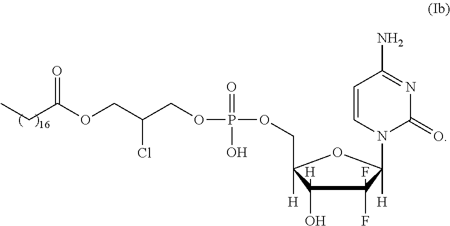

Embodiment 8. The compound according to embodiment 1, wherein R² is —CO—R³.

Embodiment 9. The compound according to embodiment 8, wherein R¹ is unsubstituted C₁₂-C₂₄ alkyl.

Embodiment 10. The compound according to embodiment 9 with structure of Formula (Ic):

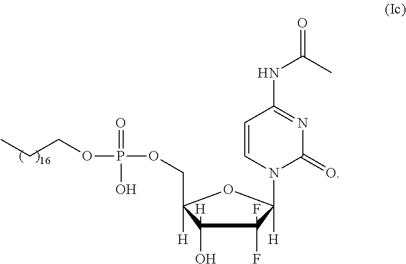

Embodiment 11. The compound according to embodiment 8, wherein R¹ is hydrogen.

Embodiment 12. The compound according to embodiment 11 with structure of Formula (Id):

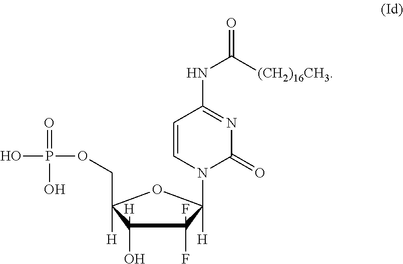

Embodiment 13. A nanoparticle composition comprising a compound having the structure:

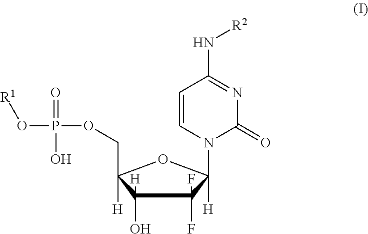

or pharmaceutically acceptable salt thereof, wherein R¹ is hydrogen, unsubstituted C₁₂-C₂₄ alkyl, or substituted or unsubstituted C₁₂-C₂₇ heteroalkyl; R² is hydrogen or —CO—R³; and R³ is substituted or unsubstituted C₁-C₂₄ alkyl.

Embodiment 14. The nanoparticle composition according to embodiment 13, said compound having the structure:

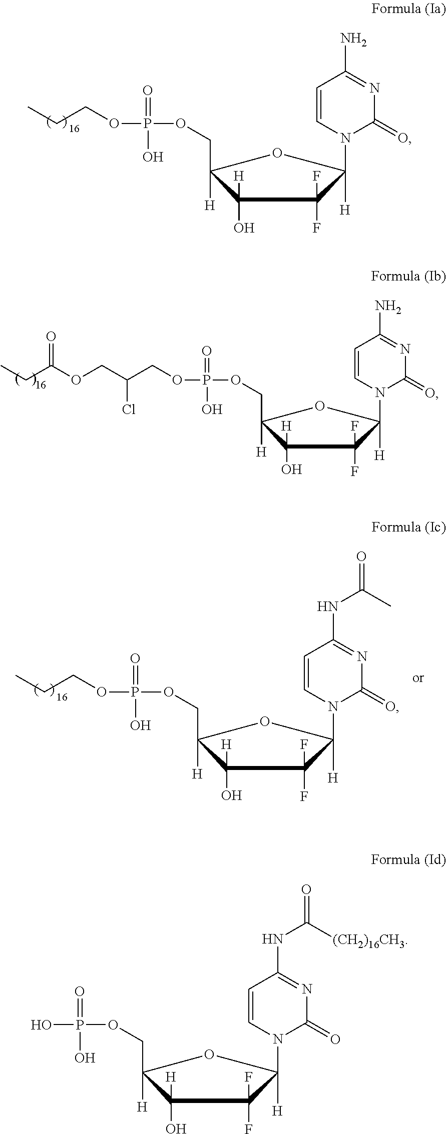

Embodiment 15. A pharmaceutical composition comprising a compound with structure of Formula (I):

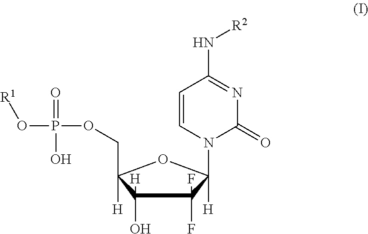

or pharmaceutically acceptable salt thereof, wherein R¹ is hydrogen, unsubstituted C₁₂-C₂₄ alkyl, or substituted or unsubstituted C₁₂-C₂₇ heteroalkyl; R² is hydrogen or —CO—R³; and R³ is substituted or unsubstituted C₁-C₂₄ alkyl; and a pharmaceutically acceptable excipient.

Embodiment 16. The pharmaceutical composition according to embodiment 15, said compound having the structure:

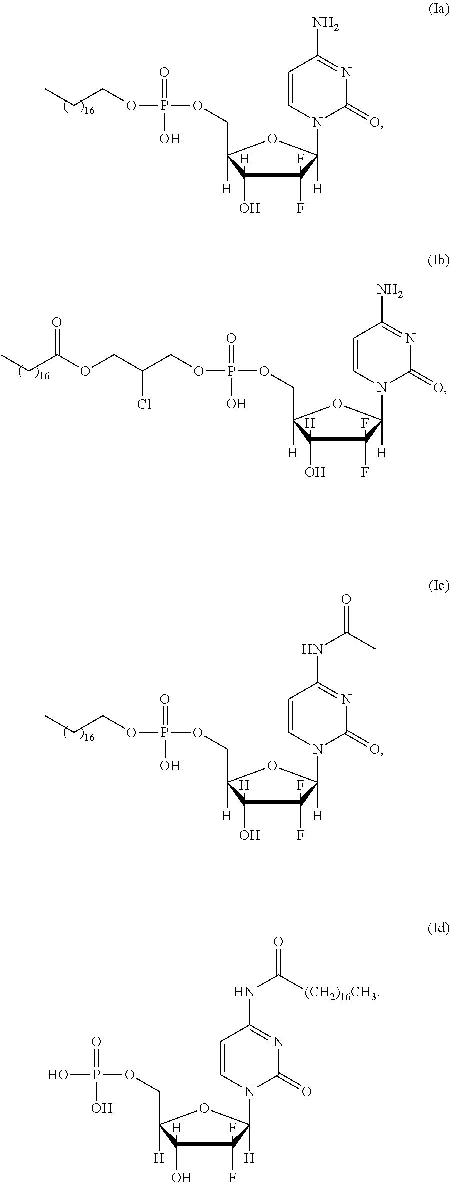

Embodiment 17. The pharmaceutical composition according to any one of embodiments 15 to 16, wherein said compound is present as a nanoparticle composition.

Embodiment 18. The pharmaceutical composition according to any one of embodiments 15 to 17, wherein said pharmaceutical composition is formulated for oral or intravenous delivery.

Embodiment 19. A method of treating cancer or a viral infection in a subject in need thereof, said method comprising administering to the subject a therapeutically effective amount of a compound according to any one of embodiments 1 to 12, a nanoparticle composition according to any one of embodiments 13 to 14, or a pharmaceutical composition according to any one of embodiments 15 to 18.

Embodiment 20. The method according to embodiment 19, wherein said pharmaceutical composition is an oral pharmaceutical composition or an intravenous pharmaceutical composition.

Embodiment 21. The method according to any one of embodiments 19 to 20, wherein said administering is oral administration or intravenous administration.

What is claimed is:

1. A compound with structure of Formula (I): 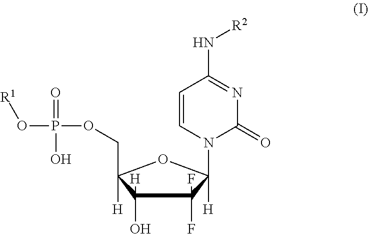
or pharmaceutically acceptable salt thereof,
wherein
R¹ is hydrogen, unsubstituted C₁₂-C₂₄ alkyl, or substituted or unsubstituted C₁₂-C₂₇ heteroalkyl;
R² is hydrogen or —CO—R³; and
R³ is substituted or unsubstituted C₁-C₂₄ alkyl.

2. The compound according to claim 1, wherein R² is hydrogen.

3. The compound according to claim 1, wherein R¹ is unsubstituted C₁₂-C₂₄ alkyl.

4. The compound according to claim 3 with structure of Formula (Ia):

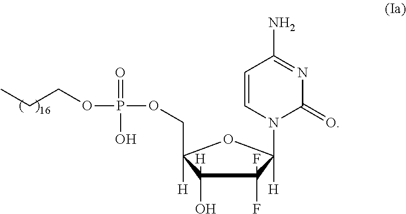

5. The compound according to claim 1, wherein R¹ is substituted or unsubstituted C₁₂-C₂₇ heteroalkyl.

6. The compound according to claim 5,
wherein
R¹ is R⁴—CO—O-L¹-;
R⁴ is substituted or unsubstituted C₁₀-C₂₀ alkyl; and
L¹ is substituted or unsubstituted C₁-C₆ alkylene.

7. The compound according to claim 6 with structure of Formula (Ib):

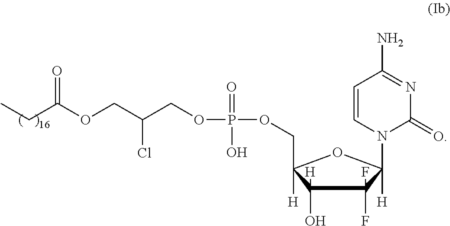

8. The compound according to claim 1, wherein R² is —CO—R³.

9. The compound according to claim 8, wherein R¹ is unsubstituted C₁₂-C₂₄ alkyl.

10. The compound according to claim 9 with structure of Formula (Ic):

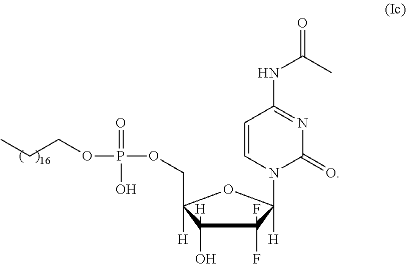

11. The compound according to claim 8, wherein R¹ is hydrogen.

12. The compound according to claim 11 with structure of Formula (Id):

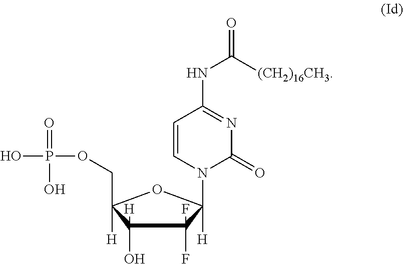

13. A nanoparticle composition comprising a compound having the structure: 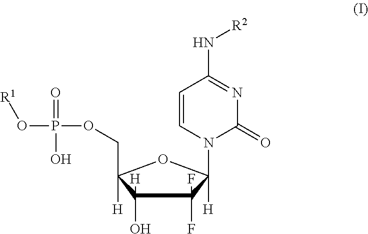
or pharmaceutically acceptable salt thereof,
wherein
R¹ is hydrogen, unsubstituted C₁₂-C₂₄ alkyl, or substituted or unsubstituted C₁₂-C₂₇ heteroalkyl;
R² is hydrogen or —CO—R³; and
R³ is substituted or unsubstituted C₁-C₂₄ alkyl.

14. The nanoparticle composition according to claim 13, said compound having the structure:

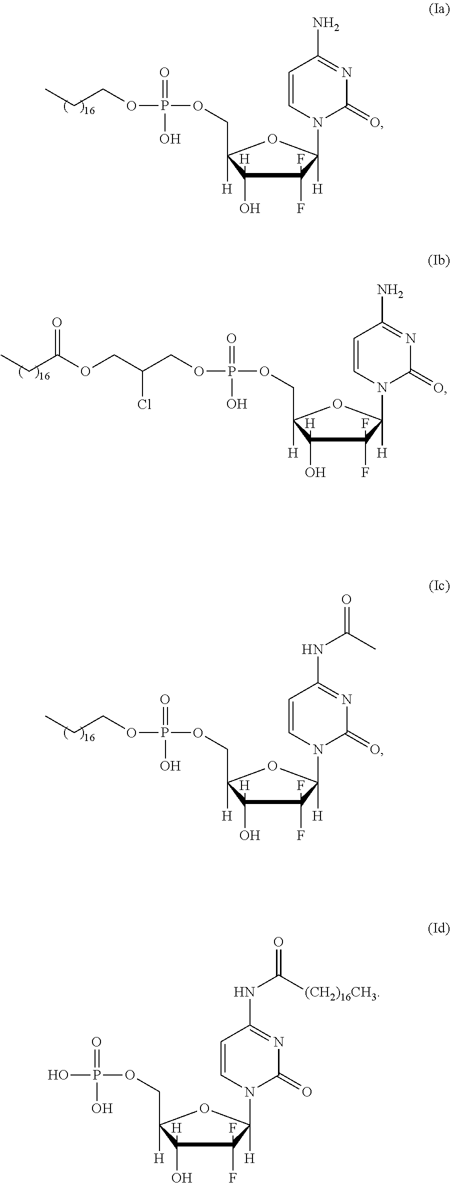

15. A pharmaceutical composition comprising a compound with structure of Formula (I): 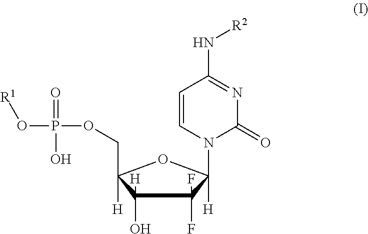
or pharmaceutically acceptable salt thereof,
wherein
R¹ is hydrogen, unsubstituted C₁₂-C₂₄ alkyl, or substituted or unsubstituted C₁₂-C₂₇ heteroalkyl;
R² is hydrogen or —CO—R³; and
R³ is substituted or unsubstituted C₁-C₂₄ alkyl; and
a pharmaceutically acceptable excipient.

16. The pharmaceutical composition according to claim 15, said compound having the structure:

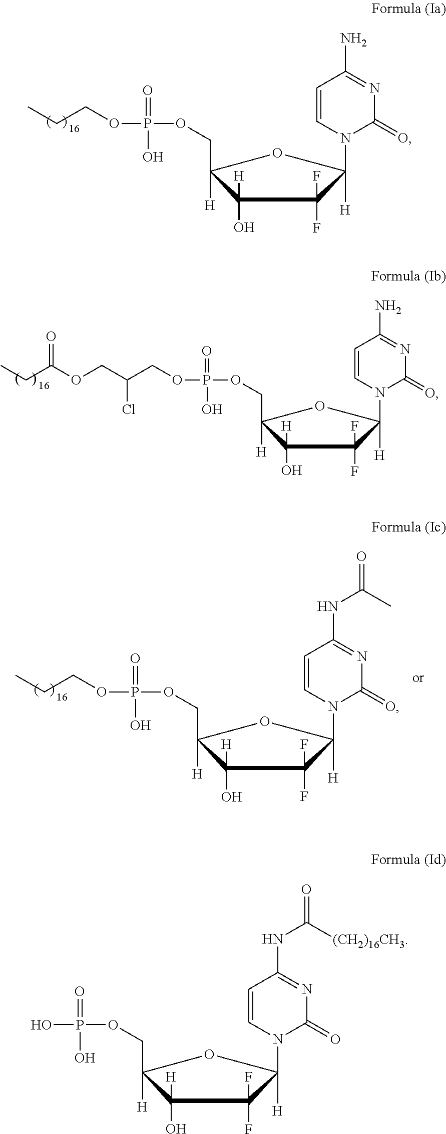

17. The pharmaceutical composition according to claim 15, wherein said compound is present as a nanoparticle composition.

18. The pharmaceutical composition according to claim 15, wherein said pharmaceutical composition is formulated for oral or intravenous delivery.

19. A method of treating cancer or a viral infection in a subject in need thereof, said method comprising administering to the subject a therapeutically effective amount of a pharmaceutical composition according to claim 15.

20. The method according to claim 19, wherein said pharmaceutical composition is an oral pharmaceutical composition or an intravenous pharmaceutical composition.

21. The method according to claim 20, wherein said administering is oral administration or intravenous administration.
